(19) United States
(12) Patent Application Publication
(10) Pub. No.: US 2006/0246037 A1
(43) Pub. Date: Nov. 2, 2006

(54) Neuroprotective effects of atf6

(76) Inventors: Bryon Zhao, Millbrae, CA (US); Mei Yu, San Francisco, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/575,457

(22) PCT Filed: Jul. 30, 2004
(86) PCT No.: PCT/US04/24571

§ 371 Date: Apr. 12, 2006

Related U.S. Application Data
(60) Provisional application No. 60/491,565, filed on Aug. 1, 2003.

(51) Int. Cl. A61K 48/00; A61K 31/045

(52) U.S. Cl. 424/93.2; 514/44; 514/738

(57) ABSTRACT

The present invention relates to methods for preventing cell death in a subject and the application of these methods for treating neurodegenerative diseases, such as Parkinson's disease, Alzheimer's disease, etc. In one embodiment of the invention, a method for preventing cell death may comprise effecting overexpression of an active form of ATF6 in the cell.

### CROSS REFERENCE TO RELATED APPLICATIONS

This application claims the benefit of U.S. Provisional Application No. 60/491,565, filed Aug. 1, 2003, which is herein incorporated by reference in its entirety.

### FIELD OF THE INVENTION

The present invention is related generally to methods for preventing cell death by the administration of a functional form of ATF6.

### BACKGROUND OF THE INVENTION

Parkinson's Disease

Parkinson's disease (PD) is one of the most common neurodegenerative diseases, with a prevalence of about 1% in people 65 years of age but increasing to 4%-5% by age 85. The major clinical symptoms of Parkinson's disease include bradykinesia, resting tremor, rigidity, and postural instability. The underlying pathology of the disease is a profound reduction in striatal dopamine content caused by the death of dopaminergic neurons in the substantia nigra (SN) pars compacts. Formo, J. Neuropathol. Exp. Neurol. 55:259-272 (1996). Disease symptoms appear after degeneration of more than 70-80% of the dopaminergic neurons in the substantia nigra.

Broadly speaking, the disease falls into two categories: late onset and early onset PD. Late onset PD is observed predominantly in patients over the age of 55 and is believed to be mostly the product of environmental factors. Late onset PD leads to dopaminergic neuron death at a faster rate, and to a more severe degree, than in healthy individuals. Early onset Parkinson's disease is less frequent, with symptoms developing between the ages of 35 and 60.

PD's distinct pathological lesions of the SN include eosinophilic filamentous inclusions known as Lewy bodies (LBs) and dystrophic neuritis, termed Lewy neurites (LNs). Id. The major components of LBs are α-synuclein and ubiquitin. Over the last few years, two mutations in α-synuclein have been found to be associated with familial cases of PD, highlighting the importance of α-synuclein and LBs in the pathogenesis of PD. Polymeropoulos et al., Science 276:2045-2047 (1997).

At present, the most common therapies for PD involve attempts to increase the dopamine content in patients via the administration of L-DOPA (as a precursor of dopamine), dopamine agonists, or monoamino oxidase B inhibitors (blocking the degradation of dopamine). These treatments alleviate some of the disease symptoms, but, because dopamineric neurons continue to die, they do not prevent the progression of the disease. At present, there are no prophylactic therapies available to stop the progression of this degenerative disease.

Parkin Gene

Lately, Autosomal Recessive Juvenile Parkinsonism (ARJP), a form of familial PD, has been linked to mutations in the human chromosome locus 6q25.2-27 (“PARK 2”gene locus, parkin gene). Matsumine et al., Am. J. Hum. Genet. 60:588-596 (1997). Neuropathological examination of patients with a mutation in this gene demonstrated that neuronal loss and gliosis were restricted to the SN and locus ceruleus, even though in most cases Lewy pathology is not observed. Mori et al., Neurology 51:890-892 (1998); Hayashi et al., Mov. Disord. 15:884-888 (2000). A recent study, however, reports a patient with a mutation in the parkin gene who also developed Lewy pathology. Farrer et al., Ann. Neurol. 50:293-300 (2001).

The parkin gene contains 12 exons spanning more than 500 kb and encodes a protein of the same name containing 465 amino acids (molecular weight of 51,652 Da). The parkin protein is homologous to ubiquitin at the N-terminus and at a RING finger-like motif at the C-terminus. Large scale genetic epidemiological studies indicate that 40-50% of familial cases of Parkinsonism include mutations in parkin. Lucking et al., N. Engl. J. Med. 342:1560-1567 (2000).

Biochemically, parkin has been found to be involved in the ubiquitin proteasome pathway of protein degradation. Shimura et al., Nat. Genet. 25:302-305 (2000); Zhang et al., Proc. Natl. Acad. Sci. USA 97:13354-13359 (2000).

The Ubiquitin Proteasome Pathway

Ubiquitin is believed to have multiple roles in cellular function, including the mediation of various stress responses, repair of damaged DNA, regulation of differential gene expression, modification of histones and receptors, and even control of the cell cycle. Other functions of ubiquitin include its chaperone-like role in the assembly of ribosomal proteins and in response to heat shock.

Ubiquitin also plays a critical role in selective protein degradation. In particular, the attachment of ubiquitin monomers to a protein earmarks that protein for degradation (ubiquitination) in what is called the ubiquitin proteasome pathway.

Polyubiquitin chains are the principal signal for targeting substrates to the 26S proteasome. Studies of structurally defined polyubiquitylated model substrates have shown tetraubiquitin to be the minimum signal for efficient proteasomal targeting of a protein. Thrower et al., EMBO J 19:94-102 (2000). Thus, a protein targeted for degradation will have at least four ubiquitin monomers attached to its surface in order to form a polyubiquitin structure having a specific three-dimensional conformation. It is believed that a hydrophobic region produced by this polyubiquitin conformation is important for the specific recognition of the degradation signal by the 26S proteasome. Beal et al., Biochemistry 37(9):2925 (1998).

Ubiquitin attachment occurs most often in three enzyme-catalyzed steps, although in a few cases ubiquitination is a two-step process. Ubiquitin is first activated by attachment to the ubiquitin-activating enzyme, E1. After an exchange reaction, ubiquitin is transferred to one of several ubiquitin-conjugating enzymes, E2. In some instances, ubiquitin is transferred directly from E2 to the target protein. In most cases, however, ubiquitin is transferred to a ubiquitin ligase, E3, which catalyzes the final attachment of ubiquitin to the target protein. The specificity of the system is conferred primarily by the E3 ligase, of which four different classes have been identified.

Recent studies have demonstrated that parkin is an E3 ligase. Imai et al., Cell 105:891-902 (2001). Therefore, when parkin mutations undermine parkin's ability to mediate the degradation of its substrate proteins, accumulation of these proteins occurs in the cell.

ATF6 and the Unfolded Protein Response

Protein degradation via the ubiquitin proteasome pathway is important in the regulation of cell activity. For example, rapid degradation of regulatory proteins allows the cell to control processes such as DNA repair, cell division, cellular differentiation, etc. Additionally, the ubiquitin proteasome pathway is involved in the degradation of proteins that have failed to fold or assemble correctly in the endoplasmic reticulum (ER).

In general, as unfolded proteins accumulate in the ER, a stereotypical response, termed the Unfolded Protein Response (UPR), is mounted as an attempt by the stressed cells to eliminate the unfolded proteins. The UPR is an intracellular signaling pathway that relays signals from the lumen of the ER in the presence of unfolded proteins to activate target genes in the nucleus. If the attempt to eliminate unfolded proteins fails, the accumulation of these proteins activates additional stress pathways that ultimately lead to cell death.

Induction of the UPR in human cells requires activation of both hIre1 and ATF6. van Laar et al., Current Protein and Peptide Science 2:169-190 (2001). hIre1 is a transmembrane protein kinase whose nuclease domain modifies a mRNA that encodes a transcription factor involved in the activation of UPR-responsive genes. Although the substrate mRNA for hIre1 has not been identified, it has been proposed that the substrate is the mRNA encoding ATF6. Id.

ATF6 is a membrane-bound transcription factor that also activates genes associated with the UPR. Ye, J. et al., Mol. Cell 6:1355-1364(2000). When unfolded proteins accumulate in the endoplasmic reticulum, ATF6 is cleaved releasing its soluble N-terminal domain into the cytoplasm. Id. Cleavage of ATF6 occurs in a regulated intramembrane proteolysis (RIP) process, which is, in general, a two-step process. In the first step, the extracytoplasmic domain is cleaved leaving a cytoplasmic domain plus a transmembrane domain containing 25 amino acids or less. Next, a second protease cleaves within the transmembrane sequence liberating the cytoplasmic domain. In the case of ATF6, the first cleavage is catalyzed by site-1 protease (S1P) and the second by site-2 protease (S2P). Id.

Once the cytoplasmic domain of ATF6 enters the nucleus, it activates the transcription of at least three genes encoding chaperone proteins that act to restore the folding of proteins in the ER lumen. Haze et al, Mol. Biol. Cell 10:3787-3799 (1999). Transcription activation by ATF6 of UPR-induced genes involves binding of ATF6 to a consensus sequence called the ER-stress responsive element (ERSE). The ERSE is found in promoters of genes that are activated upon ER-stress. van Laar et al, Current Protein and Peptide Science 2:169-190 (2001) This 19-bp promoter consists of three domains: a CCAAT region and a CCACG region, separated by a 9-bp GC-rich region. van Laar et al, Current Protein and Peptide Science 2:169-190 (2001); Roy et al. Nucleic Acids Res. 27:1437-1443 (1999); Yoshida et al. J. Biol. Chem. 273:33741-749 (1998). The consensus sequence of ERSE found by Roy et al. is CCAATCGGCGGCGGCCACG (SEQ ID NO: 3). Roy et al. Nucleic Acids Res. 27:1437-1443 (1999).

Recent reports indicate that ATF6 is capable of binding specifically to oligonucleotides containing the sequence (G)(G)TGACGTGG (SEQ ID NO: 1) or the sequence (G)(G)TGACGTGA (SEQ ID NO: 2), where both sequences are different from that of the ERSE. Wang et al., J. Biol. Chem. 275:27013-27020 (2000). The nucleotides in parenthesis showed more variability among the several oligonucleotides tested. Interestingly, the 3′ half of these ATF6 consensus DNA binding sites is the complementary sequence of the low affinity binding site for the basal transcription factor YY1 found in the mammalian ERSE. Id. Furthermore, Wang et al. also found that the full cytoplasmic domain of ATF6 is not necessarily required for binding of ATF6 to the consensus DNA binding sites mentioned above (SEQ ID NO: 1 and SEQ ID NO: 2). Wang and coworkers found specific binding to sequence SEQ ID NO: 1 and SEQ ID NO: 2 by a 145 amino acid-long fragment of ATF6 containing the basic leucine zipper (bZIP) domain. Id.

It has been reported that overexpression of full length ATF6 did not significantly affect the expression of UPR-regulated genes. However, overexpression of the soluble ATF6 cytoplasmic domain did enhance expression of the molecular chaperone BiP. van Laar et al., Current Protein and Peptide Science 2:169-190 (2001). Now, we have found that overexpression of ATF6 prevents death in cells with a defect in their UPR or in their ubiquitin proteasome pathway, which defect produces an undesired accumulation of proteins in that cell.

One possible mechanism by which an undesired accumulation of proteins occurs in diseases like PD is, for example, through the presence of parkin mutations that trigger accumulation of parkin substrate proteins in the cell. This accumulation of proteins in turn activates the cell's stress system that culminates in death when the cell is unable to cope with the buildup of proteins.

Even for idiopathic PD patients, who display LBs and show no parkin mutations, sequestration of parkin into LBs could still be a mechanism that deprives the cell of functional parkin. The resulting protein accumulation may be responsible for the loss of nigral dopaminergic neurons in these patients. Shimura et al., Science, 293:263-269 (2001).

It is important to note that the present invention may also be useful in the treatment of other diseases besides PD. A number of other neurodegenerative diseases, such as Alzheimers disease, are associated with abnormal precipitation and/or aggregation of proteins. Because these varied diseases display many pathological mechanisms in common, it is likely that they share pathways that lead to aberrant aggregation and/or precipitation of proteins and which may be alleviated by the methods presented here for treatment of Parkinson's disease.

### SUMMARY OF THE INVENTION

The present invention relates to methods for preventing cell death. In general, the methods of the invention may be practiced in vivo or in vitro. When practiced in vivo, the methods of the invention provide effective treatment for the condition suffered by a subject. When practiced in vitro, the methods of the invention provide a better understanding of the particular disease in question (Parkinson's disease, Alzheimers disease, etc.), which will allow improved diagnosis and treatment of these diseases.

In one embodiment of the invention, a method for preventing cell death comprises increasing the amount of a functional form of ATF6 in the cell.

In another embodiment of the invention, a method for preventing cell death comprises effecting overexpression of a functional form of ATF6 in the cell.

In another embodiment of the invention, the method for effecting overexpression of a functional form of ATF6 preventing cell death comprises introducing a vector comprising a nucleic acid sequence encoding ATF6 into a cell by methods known in the art. This nucleic acid sequence may be operably linked to one or more transcriptional regulatory sequences, such as promoters, enhancers, activators, terminators, polyadenylation signals, and other regulatory sequences known to the skilled artisan.

In another embodiment of the invention, the nucleic acid sequence encoding a functional form of ATF6 encodes the full-length form of ATF6. In another embodiment of the invention, the nucleic acid sequence encoding a functional form of ATF6 encodes only the N-terminal domain of AFT6.

In another embodiment of the invention, the nucleic acid sequence encoding a functional form of ATF6 encodes a functional derivative of either the full-length form of ATF6 or the N-terminal domain of ATF6. Such derivatives may be produced by techniques known in the art, including, but not limited to, deletions, additions, conservative amino acid substitutions, or other manipulations that produce a molecule that retains at least one desired biological function of ATF6 (e.g., prevention of cell death). Amino acid substitutions made in order to prepare derivatives of ATF6 are accomplished by selecting substitutions that do not differ significantly in their effect on maintaining, for example, (a) the structure of the ATF6 peptide backbone in the area of the substitution, (b) the charge or hydrophobicity of ATF6 at the target site, or (c) the bulk of a side chain. Additionally, one or more amino acids can be deleted or added from either the N-terminus or C-terminus of ATF6 (or both termini) without substantial loss of biological function.

Wang et al. reported specific binding of a fragment of ATF6 containing the bZIP domain to an ATF6-specific DNA sequence. Wang et al., J. Biol. Chem. 275:27013-27020 (2000). Because binding to this sequence by ATF6 is believed to activate transcription of UPR-inducible genes, one embodiment of a nucleic acid sequence encoding a functional form of ATF6 is a nucleic acid sequence encoding the bZIP domain of ATF6 or a functional derivative of the bZIP domain of ATF6.

In another embodiment of the invention, a method for preventing cell death comprises introducing into a cell a vector comprising a nucleic acid sequence encoding a basal transcription factor that binds to the ER-stress responsive element (ERSE), wherein this vector optionally comprises a nucleic acid sequence encoding a functional form of ATF6. Because the ERSE is part of the promoter region of UPR-inducible genes, the presence of basal transcription factors may enhance the transcription of these genes by ATF6, which is also an ERSE-binding transcription factor.

In another embodiment of the invention, the basal transcription factor that binds to the ER-stress responsive element may be NF-Y, YY1, a functional derivative of NF-Y or YY1, or any combination thereof, wherein the vector optionally comprises a nucleic acid sequence encoding a functional form of ATF6.

In another embodiment of the invention, a method for preventing cell death comprises any method known in the art for inducing the expression of ATF6. Induction of the expression of ATF6 may occur, for instance, by inducing the UPR at any point along the signaling pathway between detection of the presence of unfolded proteins in the ER and the expression of ATF6. For example, the expression of ATF6 may be induced by activating the transmembrane kinase Ire1, which has been shown to catalyze the splicing of ATF6 mRNA resulting in a mRNA fragment that encodes only the soluble domain of ATF6. Activation of Ire1 occurs after cleavage of the kinase-nuclease domain of the enzyme by presenilin 1 and 2. Therefore, the expression of ATF6 may be induced by introducing into a cell a vector comprising a nucleic acid sequence encoding one or more molecules chosen from: Ire1, presenilin 1, and presenilin 2.

In another embodiment of the invention, a method for inducing the expression of ATF6 comprises introducing into a cell a vector containing a nucleic acid sequence encoding a transcription factor of ATF6.

In another embodiment of the invention, a method for preventing cell death comprises introducing into a cell a vector comprising a nucleic acid sequence encoding either site-1 protease (SIP), site-2 protease (S2P), or both; wherein the vector optionally comprises a nucleic acid sequence encoding a functional form of ATF6. These nucleic acid sequences may be operably linked to one or more transcriptional regulatory sequence.

In another embodiment of the invention, a method for preventing cell death comprises introducing into a cell a vector comprising a nucleic acid sequence comprising a promoter and/or an enhancer that replaces the original promoter of the gene encoding ATF6 in the cell's genome. Incorporation of the nucleic acid sequence coding for the promoter and/or enhancer into the cell genomic DNA may occur, for example, by homologous recombination.

In another embodiment of the invention, a vector comprising any of the nucleic acid sequences useful in the methods of the invention is incorporated into the genome of a cell by nonhomologous recombination.

Another embodiment of the invention relates to compositions suitable for preventing cell death.

In another embodiment of the invention, the composition comprises a nucleic acid encoding a functional form of ATF6. Compositions optionally comprise appropriate pharmaceutically acceptable additives, lubricants, diluents, buffers, moistening agents, preservative agents, flavorings, adjuvants, carriers, stabilizers, suspending agents, emulsifying agents, propellants, and/or other vehicles.

In another embodiment of the invention, the composition comprises a nucleic acid coding for a molecule that regulates the concentration of a parkin-interacting agent in the cell, such as, for example, antisense nucleic acids.

Another embodiment of the invention relates to methods to screen for substances that affect ATF6 activity. In one embodiment, the method can determine whether a substance increases or decreases the activity of at least one functional form of ATF6 in a cell whose production by a cell evokes a responsive change in at least one characteristic. This method can comprise providing two cell lines, one which produces at least one functional form of ATF6 and a second which produces the at least one functional form of ATF6 at a lower level than the first cell line, or does not produce the at least one functional form of ATF6 at all. Then, the substance can be placed in contact with the first and second cell lines. Finally, one can compare the at least one characteristic of the first cell line with the second cell line.

Additional objects and advantages of the invention will be set forth in part in the detailed description that follows, and in part will be apparent upon considering the application as a whole. Additionally, further objects and advantages of the invention may also be learned by practicing the invention.

### BRIEF DESCRIPTION OF THE DRAWINGS

FIG. 1. Amino acid sequence of human ATF6-α (Swiss-Prot accession number P18850); SEQ ID NO: 4.

FIG. 2. Amino acid sequence of human ATF6-β (Swiss-Prot accession number Q99941); SEQ ID NO: 5.

FIG. 3. Amino acid sequence of murine ATF6-α; SEQ ID NO: 6.

FIG. 4. Amino acid sequence of murine ATF6-β (Swiss-Prot accession number 035451); SEQ ID NO: 7.

FIG. 5. DNA sequence of human ATF6-α (GenBank accession number NM_{—}007348); SEQ ID NO: 8.

FIG. 6. DNA sequence of human ATF6-β (GenBank accession number NM_{—}004381); SEQ ID NO: 9.

FIG. 7. DNA sequence of murine ATF6-α (GenBank accession number AK087000); SEQ ID NO: 10.

FIG. 8. DNA sequence of murine ATF6-β (GenBank accession number AK090378); SEQ ID NO: 11.

### DETAILED DESCRIPTION OF THE INVENTION

The embodiments described and the terminology used herein are for the purpose of describing exemplary embodiments only, and are not intended to be limiting. The scope of the present invention is intended to encompass additional embodiments not specifically described herein, but that would be apparent to one skilled in the art upon reading the present disclosure and practicing the invention.

Other than in the operating examples, or where otherwise indicated, all numbers expressing quantities of ingredients, reaction conditions, and so forth used in this application are to be understood as being modified in all instances by the term “about.” Accordingly, unless the contrary is indicated, the numerical parameters set forth in this application are approximations that may vary depending upon the desired properties sought to be obtained by the present invention. At the very least, and not as an attempt to limit the application of the doctrine of equivalents to the scope of the claims, each numerical parameter should be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, exemplary methods and materials are described for illustrative purposes. All publications mentioned in this application are incorporated by reference to disclose and describe the methods and/or materials in connection with which the publications are cited.

Additionally, the publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that as used herein and in the appended claims, the singular forms “a”, “and”, and “the” include plural referents unless the context clearly dictates otherwise. Thus, for example, reference to “an antibody” includes a plurality of such antibodies and reference to “a vector” includes reference to one or more vectors and equivalents thereof known to those skilled in the art.

Methods, techniques, and/or protocols (collectively “methods”) that can be used in the practice of the invention are not limited to the particular examples of these procedures cited throughout the specification but embrace any procedure known in the art for the same purpose. For example, with respect to methods for the delivery of nucleic acids to the brain, the present invention is not limited to the protocols cited herein, but includes any method available in the art to the skilled artisan to deliver nucleic acids to brain cells.

Furthermore, although some methods may be described in a particular context in the specification, their use in the instant invention is not limited to that context. For example, methods to prepare functional derivatives of a molecule will be described in the context of derivatives of ATF6 but these methods are equally applicable to the preparation of derivatives of any other molecule useful in the methods of the invention.

### Definitions

The terms “nucleic acid” or “polynucleotide” refer to a polymer of at least two nucleotides. The term nucleic acid includes deoxyribonucleic acid (DNA) and ribonucleic acid (RNA). RNA includes, but is not limited to, tRNA (transfer RNA), snRNA (small nuclear RNA), rRNA (ribosomal RNA), mRNA (messenger RNA), anti-sense RNA, and ribozymes. Similarly, DNA includes, but is not limited to, plasmid DNA, viral DNA, linear DNA, chromosomal DNA, or derivatives of these groups. In addition, these forms of DNA and RNA may be single, double, triple, or quadruple stranded. The term nucleic acid also includes PNAs (peptide nucleic acids), phosphorothioates, and other variants of the phosphate backbone of native nucleic acids. In some cases, when a nucleic acid encodes a polypeptide to be introduced into a cell, it may be advantageous, although not necessary, that such nucleic acid be in the form of a double-stranded cDNA lacking intron segments.

A nucleic acid may be constructed to express a whole or partial protein. Once a polynucleotide has been delivered to a cell, the nucleic acid can remain separate from the endogenous genetic material. Alternatively, the nucleic acid could recombine with (become a part of) the endogenous genetic material. For example, DNA can be inserted into chromosomal or genomic DNA by either homologous or nonhomologous recombination. Homologous recombination is described, for example, in U.S. Pat. Nos. 5,282,071, and 5,578,461. Nonhomologous recombination is described, for example, in PCT Application No. WO 00/49162.

Optionally, depending on their particular function, nucleic acid molecules may be operably linked to one or more transcriptional regulatory sequences.

One embodiment of the term vector refers to a polynucleotide having a nucleotide sequence that can assimilate other nucleic acid sequences, and propagate those sequences in an appropriate host. A vector may be capable of self-replication or may simply serve as carrier for a given nucleic acid sequence. Vectors may originate from viruses, plasmids, or the cells of a higher organism, and often contain DNA sequences from several sources. Examples of vectors include plasmids, cosmids, and yeast artificial chromosomes. Examples of viral vectors include adenovirus vectors, adenoassociated viral vectors (MV), and retrovirus vectors. Non-viral vectors include expression plasmid vectors typically used in mammalian cells, and are exemplified by pBK-CMV, pCAGGS, pcDNA3.1, pZeoSV, and the like. See, e.g., European Patent Application Nos. 1 122 312 and 1 132 098.

Another embodiment of the term vector refers to a carrier used to introduce a nucleic acid sequence into a cell. This type of vector may form complexes with the nucleic acid sequence, for example, an antisense oligonucleotide complexed to protein, an antisense oligonucleotide complexed with lipid-based nucleic acid transduction systems, or other non-viral carrier systems. Suitable lipids and related analogs are described in U.S. Pat. Nos. 5,208,036, 5,264,618, 5,279,833, and 5,283,185. Vectors can also be particulate carriers, examples of which include polymethyl methacrylate polymers and polylactides and poly(lactide-co-glycolides). See, e.g., McGee et al., J. Micro Encap. (1996).

Several non-viral methods for the transfer of polynucleotides into cultured mammalian cells are also contemplated by the present invention, and include, without being limited to, calcium phosphate precipitation; DEAE-dextran; electroporation; direct microinjection; DNA-loaded liposomes; and receptor-mediated transfection. These techniques are disclosed in, e.g., PCT patent application WO 01/42451. Some of these techniques may be successfully adapted for in vivo or ex vivo use.

Vectors are normally used to mediate the expression of a molecule of interest in a cell, in which case they are called expression vectors. An “expression vector” is a vector in which the nucleic acid sequence encoding a molecule of interest is operably linked to suitable transcriptional regulatory sequences capable of effecting the expression of the molecule of interest in a cell.

A nucleic acid is operably linked when it is placed into a functional relationship with another nucleic acid sequence. For example, a promoter or enhancer is operably linked to a coding sequence if it affects the transcription of the sequence.

A transcriptional regulatory sequence is a nucleic acid sequence that regulates the expression of a nucleic acid sequence to which it is operably linked. A regulatory sequence may include elements that are naturally responsible for expressing a particular nucleic acid or may include sequences of a different origin. In general, regulatory sequences can be sequences of eukaryotic or viral genes or derived sequences that stimulate or repress transcription of a gene in a specific or non-specific manner and in an inducible or non-inducible manner. Transcriptional regulatory sequences include, e.g., promoters, origins of replication, RNA splice sites, enhancers, transcriptional termination sequences, signal sequences which direct the polypeptide into the secretory pathways of the target cell. As the skilled artisan will appreciate, regulatory sequences may be constitutive or inducible depending on the particular nature of the sequence and the role of the nucleic acid sequence operably linked to the regulatory sequence.

A promoter is a transcriptional regulatory sequence that directs RNA polymerase to bind to DNA and initiate RNA synthesis. A strong promoter is one that causes mRNAs to be initiated at high frequency. Examples of promoters that can be used in the construction of vectors for the delivery of nucleic acid sequences to neuronal tissue include the promoter/enhancer from the human cytomegalovirus (hCMV) (WO 98/46273), and the simian virus 40 (SV40) promoter (Harada et al., Cancer Gene Ther. 7(5):799-805 (2000). Other promoters that cause expression in the brain include the platelet-derived growth factor (PDGF) promoter (WO 02/26936); prion promoter (Hsiao et al., Science 274:99-102 (1996); and the neuron-specific enolase promoter (Xu R., et al. Gene Ther. 8(17):1323-32 (2001).

An enhancer is a transcriptional regulatory sequence that increases transcription by a promoter. Enhancers can effectively increase transcription when located either 5′ or 3′ to the transcription unit. They are also effective if located within an intron or within the coding sequence itself. Typically, viral enhancers may be used, including SV40 enhancers, cytomegalovirus enhancers, polyoma enhancers, and adenovirus enhancers. See, e.g., WO 98/55616 and U.S. Pat. Nos. 6,248,555 and 6,323,030. Enhancer sequences from mammalian systems are also commonly used, such as the mouse immunoglobulin heavy chain enhancer. See, e.g., WO 92/04440.

A transcriptional terminator sequence refers either to a sequence that provides a signal that terminates transcription by the polymerase that recognizes the selected promoter or to a signal sequence for polyadenylation. These terminator sequences may be isolated from bacteria, fungi, viruses, animals and/or plants. In the case of terminators from prokaryotic cells, the terminator generally includes a polyadenylation signal, which facilitates the addition of polyadenylate sequences to the 3′ end of a primary transcript. Examples of terminators include those from the cytomegalovirus and SV40 systems, as well as the bovine growth hormone (BGH) polyadenylation sequence. See, e.g., WO 98/55616, WO 02/16594 and U.S. Pat. Nos. 6,248,555 and 6,323,030.

Mammalian expression vector systems may include a selectable marker gene. Examples of suitable markers include the dihydrofolate reductase gene (DHFR), the thymidine kinase gene (TK), fluorescent green protein, β-galactosidase, or prokaryotic genes conferring drug resistance. The first two marker genes are normally used with mutant cell lines that lack the ability to grow without the addition of thymidine to the growth medium. Transformed cells can then be identified by their ability to grow on non-supplemented media. Examples of prokaryotic drug resistance genes useful as markers include genes conferring resistance to G418, mycophenolic acid, and hygromycin. See, e.g., U.S. Pat. No. 6,165,793

In the context of the invention, overexpression of ATF6 occurs when a functional form of ATF6 is expressed in a modified host cell at higher levels than in the original host cell. An original host cell becomes a modified host cell after any manipulation that alters the expression of any molecule in the original host cell. An example of a modification that alters the expression of ATF6 in the original host cell includes the introduction of a nucleic acid sequence encoding, for instance, ATF6 under a strong promoter.

Inducing the expression of ATF6 may be accomplished by any modification of the host cell that will result in or increase the expression of ATF6. For example, induction may be carried out by introducing a nucleic acid sequence into the host cell, wherein the nucleic acid sequence encodes transcription factors of ATF6, proteases that catalyze the cleavage of the full-length transmembrane form of ATF6, or any other molecule that directly or indirectly may initiate transcription of ATF6.

Alternatively, induction may also be effected by exposing the host cell to suitable environmental conditions. For example, environmental conditions that may induce expression of ATF6 include incubation of the cell in the presence of tunicamycin or any other inhibitor of protein folding known in the art.

The expression of an endogenous protein, for example endogenous ATF6, refers to the expression of the original protein gene in a host cell. Expression of an exogenous gene refers to the expression of a nucleic acid sequence that has been introduced into the cell. The invention contemplates, for example, altering the expression of the endogenous ATF6 gene by replacing the endogenous promoter with an exogenous promoter sequence.

A. UPR-regulated gene is a gene that codes for a protein that is involved in a cell's response to ER-stress, such as the unfolded protein response. Alternatively, these genes are also called, inter alia, UPR-induced genes or UPR-responsive genes. The promoter region of a UPR-regulated gene comprises the ERSE consensus sequence. van Laar et al., Current Protein and Peptide Science 2:169-190 (2001); Roy et al. Nucleic Acids Res. 27:1437-1443 (1999); Yoshida et al. J. Biol. Chem. 273:33741-749 (1998). An example of a UPR-regulated gene include the molecular chaperones BiP. van Laar et al., Current Protein and Peptide Science 2:169-190 (2001). However, UPR-regulated genes include a wide variety of genes that are involved in various aspects of ER function that include not only protein folding, but also protein glycosylation, secretion, degradation, and membrane biosynthesis, among others. Id. ATF6-regulated genes include, but are not limited to, UPR-regulated genes.

A therapeutically beneficial effect of a modification of an original host cell is obtained when the modified host cell shows a measurable improvement in a desired characteristic (e.g., prevention of cell death) with respect to the original host control. A therapeutically beneficial effect may come about, for example, as a result of overexpression of a given protein in the cell, for instance ATF6.

A functional form of ATF6 is any form of ATF6 whose overexpression, directly or indirectly, produces a beneficial therapeutic effect in the host cell. For the purposes of this invention, a functional form of ATF6 includes, but is not limited to, a full-length form of ATF6, an N-terminal domain of ATF6, a bZIP-ATF6 fragment of ATF6 and/or a functional derivative of any of these forms of ATF6.

Unless otherwise explicitly specified in this application, any reference to ATF6 should also be construed as a reference to the full-length form of ATF6, the N-terminal domain of ATF6, a bZIP-ATF6 fragment of ATF6, a functional derivative of the full-length form of ATF6, a functional derivative of the N-terminal domain of ATF6, a functional derivative of a bZIP-ATF6 fragment of ATF6, or any combination of any of these forms of ATF6. This construction should be applied unless the context clearly indicates that only a particular form of ATF6 is being referenced.

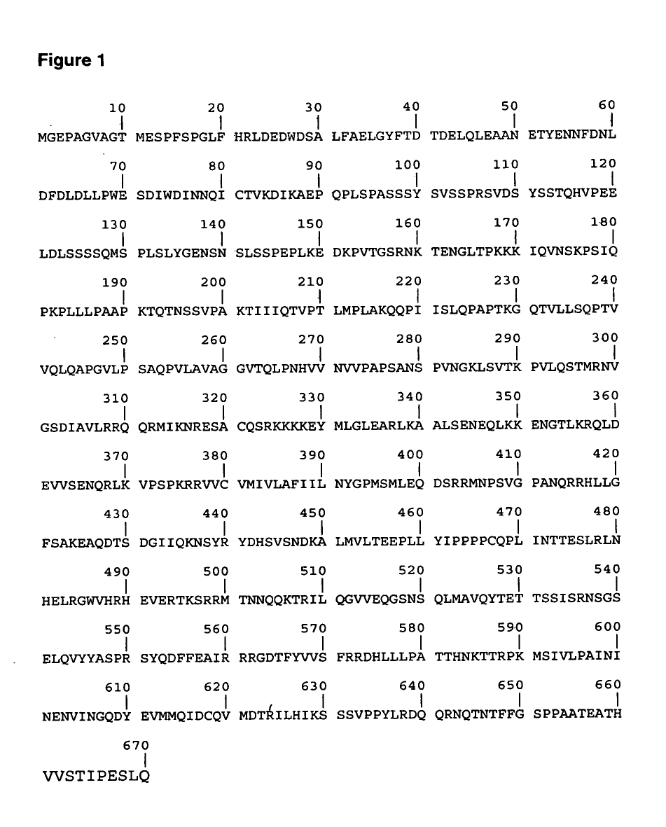

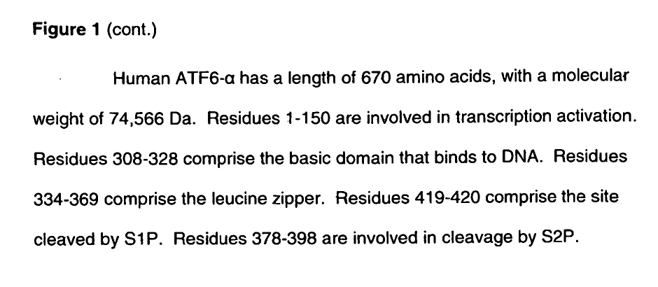

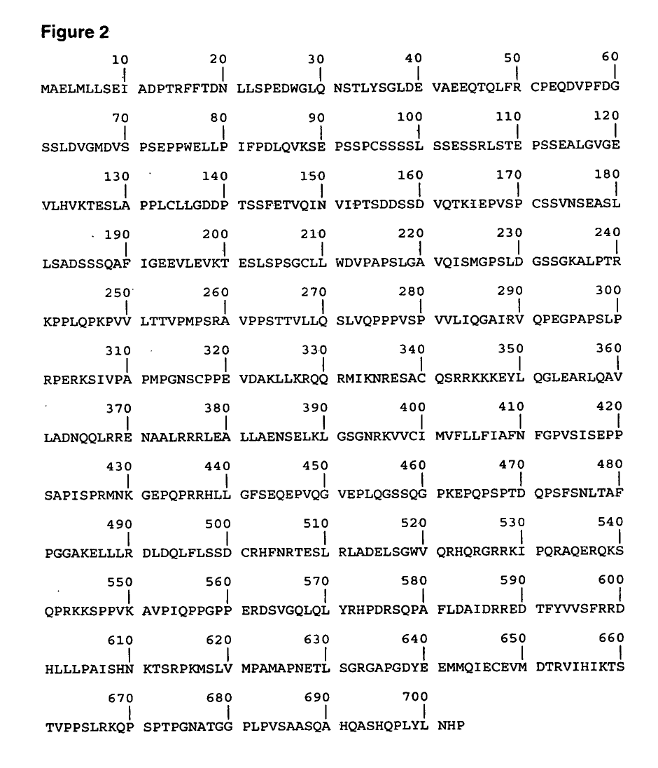

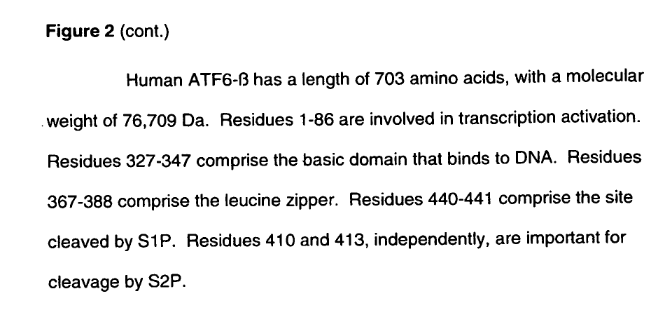

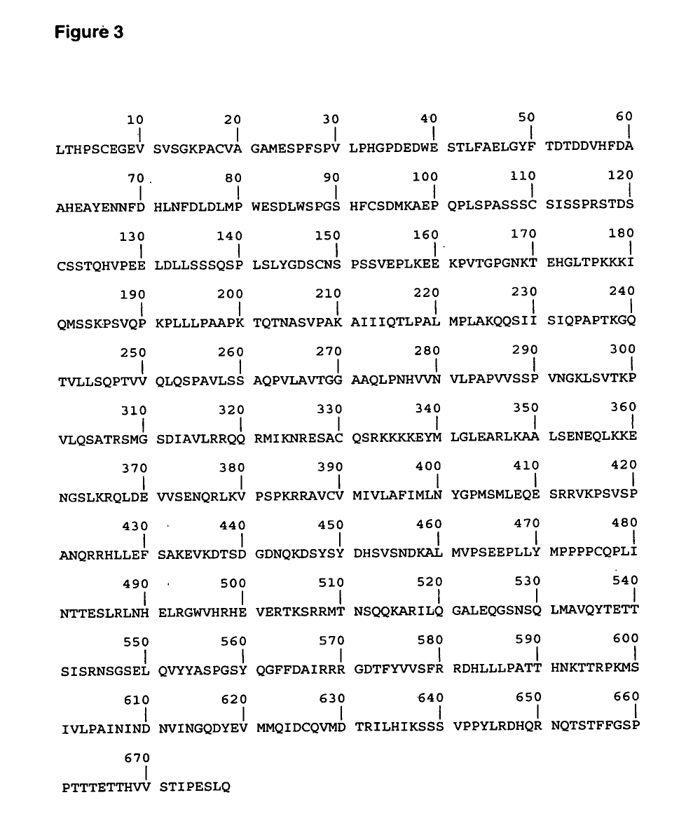

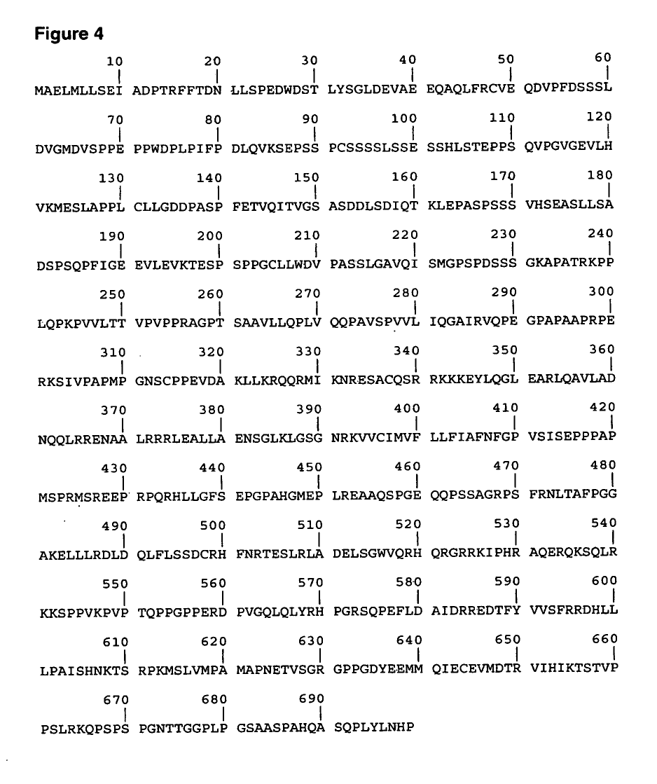

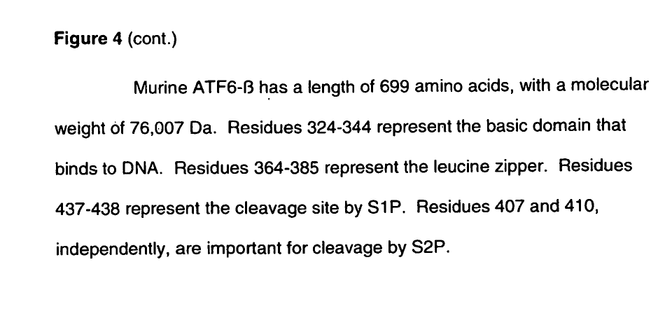

The full-length form of ATF6 is a single-pass type 2 transmembrane protein that undergoes regulated intramembrane proteolysis (RIP). Examples of the full-length form of ATF6 include the polypeptides coded by human and mouse ATF6. See FIGS. 1-4. As defined here, the N-terminal domain of ATF6, also termed the active form or mature form of ATF6, includes any of the polypeptides resulting from cleavage of the full-length form of ATF6 by either S1P or S2P. The ATF6 cleavage sites for S1P and S2P are disclosed in Ye et al (2000). Additionally, the N-terminal domain of ATF6 encompasses a bZIP-ATF6 fragment. A bZIP-ATF6 fragment is any polypeptide of ATF6 that comprises the bZIP domain of ATF6 and that is able to effect transcription of the natural target genes for which ATF6 is a transcription factor. Whether a bZIP-ATF6 fragment can effect transcription of the natural target genes for which ATF6 is a transcription factor can be determined by measuring the therapeutic effect, if any, of the overexpression in a cell of the bZIP-ATF6 fragment. If the cell shows a therapeutically beneficial effect with respect to the original host cell as shown in the methods of the invention, it can be concluded that the bZIP-ATF6 fragment can effect transcription of the natural target genes for which ATF6 is a transcription factor.

A functional derivative of a given protein is any molecule derived or obtained from that protein that retains at least one desired biological function of the original protein. For example, a functional derivative of ATF6 retains at least one desired biological function of ATF6 if the overexpression of the derivative produces a therapeutically beneficial effect in a host cell. Functional derivatives may be produced by techniques known in the art, including deletions or additions of amino acids, conservative amino acid substitutions, or other manipulations that will not affect the ability of the derivative to provide a therapeutically beneficial effect to the modified host cell.

Substitution of an amino acid may involve a conservative or nonconservative amino acid substitution. Conservative amino acid substitution means replacing an amino acid residue with another amino acid having similar chemical or physical properties (e.g., structure, size, charge, acidity or basicity, hydrophobicity, etc.) and that does not affect the ability of the derivative to retain at least one desired biological function of the original protein. Analogously, a non-conservative substitution refers to the substitution of an amino acid residue with another amino acid having dissimilar chemical or physical properties. Computer programs that predict the effect of a specific amino acid substitution or mutation on the structure of the protein are known in the art. For example, PCT Patent Application No. WO 02/543063 discloses one such method and also reviews the state of the art in this field.

A derivative may also result from the cleavage of the parent molecule, cyclisation and/or coupling with one or more additional moieties that improve solubility, altering the lipophilic characteristics to enhance uptake by cells, altering stability or biological half-life, decreasing cellular toxicity, or, in particular in vitro or ex vivo applications, acting as a label for subsequent detection, or the like. Moreover, a derivative may result from post-translational or post-synthesis modification such as the attachment of carbohydrate moieties or chemical reaction(s) resulting in structural modification(s) such as the alkylation or acetylation of amino acid residues or other changes involving the formation of chemical bonds.

Optimal alignment is defined as an alignment giving the highest percent identity score. An alignment may be optimized, for example, using a variety of commercially available sequence analysis programs, such as BLAST (Basic Local Alignment Search Tool) program available from the National Center for Biotechnology information at http://www.ncbi.nlm.nih.gov. See also Altschul, S. F. et al., “Basic local alignment search tool.” J. Mol. Biol. 215:403-410 (1990). Local alignment, as opposed to global alignment, analyzes similarities between sequences based on fragments of a longer sequence and is able to detect relationships among sequences which share only isolated regions of similarity. Therefore, local alignment is useful when analyzing sequences of different lengths or in the analysis of multiple regions of similarity within long DNA (or protein) sequences. Other local alignment programs include LALIGN using a ktup of 1, default parameters and the default PAM. The LALIGN program is found in the FASTA version 1.7 suite of sequence comparison programs. Pearson, W. R. et al., PNAS 85:2444-2448 (1988); Pearson, W. R., Methods in Enzymology 183:63-98 (1990). Computer-assisted alignment also can be performed using the CLUSTAL-W program in MacVector, operated with default parameters and a BLOSUM similarity matrix. Global sequence alignment can be performed using, for example, the FASTD13 computer program based on the algorithm of Brutlag et al. (Comp. App. Biosci. 6:237-245 (1990)).

Percent sequence identity with respect to two amino acid or polynucleotide sequences, refers to the percentage of residues that are identical in the two sequences when the sequences are optimally aligned. Thus, 80% amino acid sequence identity means that 80% of the amino acids in two or more optimally aligned polypeptide sequences are identical. If a gap needs to be inserted into a first sequence to optimally align it with a second sequence, the percent identity is calculated using only the residues that are paired with a corresponding amino acid residue (i.e., the calculation does not consider residues in the second sequences that are in the “gap” of the first sequence).

A first polypeptide or polynucleotide region is said to be homologous to a second polypeptide region when the regions are essentially co-extensive when the sequences containing the regions are aligned using a sequence alignment program, as described above. Homologous sequences are referred to as corresponding to one another.

Homologous polynucleotide or polypeptide fragments typically contain a similar, if not identical, number of residues. It will be understood, however, that corresponding fragments may contain insertions or deletions of residues with respect to one another, as well as some differences in their sequences.

The term sequence identity means nucleic acid or amino acid sequence identity in two or more aligned sequences, aligned as defined above. Sequence similarity between two polypeptides is determined by comparing the amino acid sequence and any conservative amino acid substitutions of one polypeptide to the sequence of a second polypeptide. Thus, 80% protein sequence similarity means that 80% of the amino acid residues in two or more aligned protein sequences are conserved amino acid residues, i.e. are conservative substitutions.

Hybridization includes any process by which a strand of a nucleic acid joins with a complementary nucleic acid strand through base-pairing. Thus, strictly speaking, the term refers to the ability of the complement of the target sequence to bind to the test sequence, or vice-versa.

While it is understood that the lower the salt concentration, the higher the stringency, in general, hybridization conditions are based on the melting temperature (Tm) of the nucleic acid binding complex or probe at a given ionic strength and are typically classified by the degree of stringency of the conditions under which hybridization is measured. For example, maximum stringency typically occurs at about Tm-5° C. (5° C. below the Tm of the probe) with salt concentrations in the washing solution typically being 0.1×SSC (20×SSC=3.0 M NaCl/0.3 M trisodium citrate). High stringency typically occurs at about 5-10° C. below the Tm; intermediate stringency at about 10-20° C. below the Tm of the probe; and low stringency at about 20-25° C. below the Tm. See, e.g., U.S. Pat. Nos. 6,051,385 and 6,111,090. Functionally, maximum stringency conditions may be used to identify nucleic acid sequences having identity or near-identity with the hybridization probe; while high stringency conditions may be used to identify nucleic acid sequences having about 80% or more sequence identity with the probe. Persons skilled in the art will appreciate that specific conditions will vary according to the composition of the probe, composition of the target substrate, and the like. An example of high stringency conditions for a probe having a Tm of 70° C. includes hybridization at about 65° C. in about 5×SSPE and washing at about 65° C. in about 0.1×SSPE (where 1×SSPE=0.15 M sodium chloride, 0.010 M sodium phosphate, and 0.001 M disodium EDTA). Examples of various conditions of stringency are taught in Wahl and Berger (Methods Enzymol. 152:399-407(1987)) and Kimmel (Methods Enzymol. 152:507-511 (1987)). One general guide for nucleic acid hybridization is Tijssen, “Laboratory Techniques in Biochemistry and Molecular Biology” Elsevier, vol. 24, Hybridization with Nucleic Acid Probes, Part I Overview of principles of hybridization and the strategy of nucleic acid assays (1993).

In the context of the invention, a cell in need of a method for preventing cell death is a cell in which undesired accumulation of proteins occurs. For example, such a cell may include a cell in which parkin substrates accumulate due to the inability of endogenous parkin protein to mediate their degradation. Other examples may include cells in which neurofibrillary tangles (NFT) accumulate, for instance in patients with Alzheimer's disease, etc. Similarly, a subject in need of a method for preventing cell death is a subject in which undesired accumulation of proteins causes cell death.

The terms effective dose, effective amount, therapeutically effective amount, and the like mean administering an agent in an amount sufficient to provide the desired physiological, pharmacological, and/or cognitive change. This will vary depending on the patient, the disease, and the treatment. The amount may either be a dose for the treatment in a subject believed to have a particular disorder, in which case it should sufficiently alleviate or ameliorate the symptoms of the disorder or condition, or be a prophylactic dose, in which case it should be sufficient to prevent, partially or completely, the appearance of symptoms in the subject.

The terms treatment, treating and the like are used herein to mean obtaining a desired pharmacologic and/or physiologic effect. The effect may be prophylactic in terms of completely or partially preventing a disease or a symptom thereof and/or may be therapeutic in terms of a partial or complete cure for a disease and/or an adverse effect attributable to the disease. Treatment as used herein covers any treatment of a disease in a mammal, for example a human, and includes:

(a) preventing the disease from occurring in a subject that may be predisposed to the disease but not yet diagnosed as having it;

(b) altering the progression of the disease, i.e., slowing or arresting its development; or

(c) reducing the severity of the disease, i.e., causing regression of the disease or alleviating one or more symptoms of the disease.

Abbreviations

ARJP Autosomal Recessive Juvenile Parkinsonism
bZIP Basic leucine zipper
CMV Cytomegalovirus
ER Endoplasmic reticulum
ERSE ER-stress responsive element
hIre1 A human transmembrane protein kinase
Ire1 A transmembrane protein kinase
LB Lewy body
LN Lewy neurite
NFT Neurofibrillary tangles
NF-Y A basal transcription factor that binds to the ERSE
PD Parkinson's disease
RIP Regulated intramembrane proteolysis
S1P Site 1 protease
S2P Site 2 protease
SN Substantia nigra
SOD1 Superoxide dismutase 1
UPR Unfolded Protein Response
YY1 A basal transcription factor that binds to the ERSE

### General Aspects of the Invention

The present invention relates to methods for preventing cell death. For example, preventing neuronal cell death is contemplated within the present invention, including preventing the death of nigral neurons in a mammal, including humans.

In general, the methods of the invention may be practiced in vivo or in vitro. When practiced in vivo, the methods of the invention will provide effective treatment for the condition suffered by the subject. When practiced in vitro, the methods of the invention will provide a better understanding of the particular disease in question (Parkinson's disease, Alzheimers disease, etc.) that will allow improvement of the diagnosis and treatment of these diseases.

One aspect of the present invention is based on the unexpected discovery that overexpression of ATF6 in a cell prevents cell death that would otherwise occur when an undesired accumulation of proteins occurs in that cell. For example, the invention may be useful for treating a condition such as Parkinson's disease (PD) associated with the abnormal accumulation of molecules that interact with parkin and that are not properly disposed of within a cell.

Additionally, the present invention may also be useful in the treatment of other diseases besides PD. A number of other neurodegenerative diseases are associated with abnormal precipitation and/or aggregation of proteins. For example, the brains of patients with Alzheimer's disease exhibit neurofibrillary tangles (NFT), senile plaques, and cerebrovascular deposits of amyloid-beta; the brains of patients with prion disorders exhibit plaques comprising prion proteins; the brains of patients with Huntington's disease exhibit huntingtin precipitates; patients with dominantly inherited spinocerebellar ataxias exhibit corresponding ataxin protein precipitates; patients with multiple system atrophy exhibit alpha-synuclein deposits; patients with progressive supranuclear palsy exhibit tau precipitates; and patients with familial amyotrophic lateral sclerosis exhibit SOD1 precipitates. See, e.g., Johnson, W. G., J. Anat. 4:609-616 (2000). Because these various diseases share common pathological mechanisms, it is likely that they share pathways that lead to aberrant aggregation and/or precipitation of proteins. Hardy, J. and Gwinn-Hardy, K., Science 282(5391):1075-1079 (1998).

The present invention is intended to be used as either a stand-alone therapy, or as a conjunctive therapy with other agents that are either palliative (e.g., agents that relieve the symptoms of the disorder to be treated), and/or agents that target the etiology of the disorder. For example, the administration to a subject of a composition that increases the expression of ATF6 may be carried out in conjunction with the administration of L-DOPA, dopamine agonists, monoamine oxidase B inhibitors, or any other composition useful in the treatment of a neurodegenerative disease, such as Parkinson's disease.

Any method known in the art that increases the expression of ATF6 in a neuron is part of the invention. In one embodiment of the invention, a method for preventing cell death may comprise introducing a vector comprising a nucleic acid sequence encoding a functional form of ATF6 into a cell by gene therapy methods known in the art.

Gene Therapy

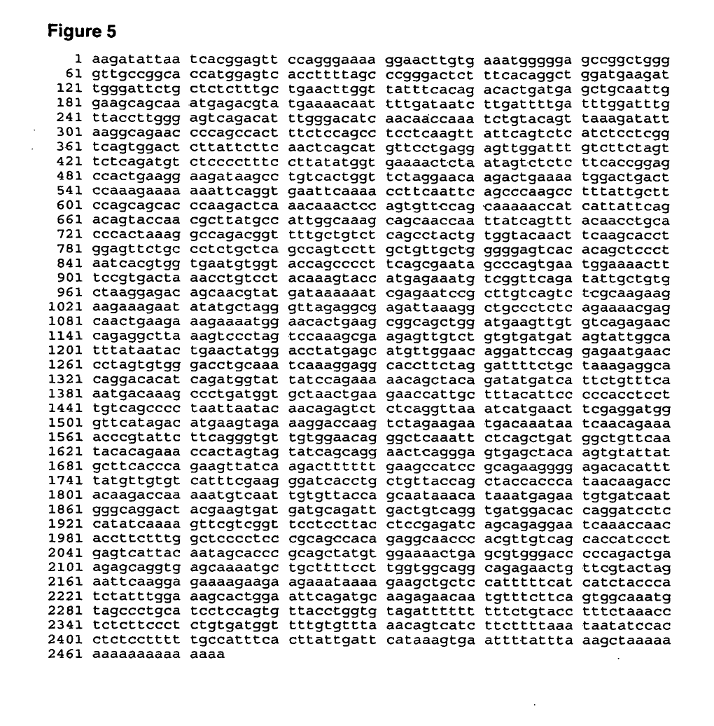

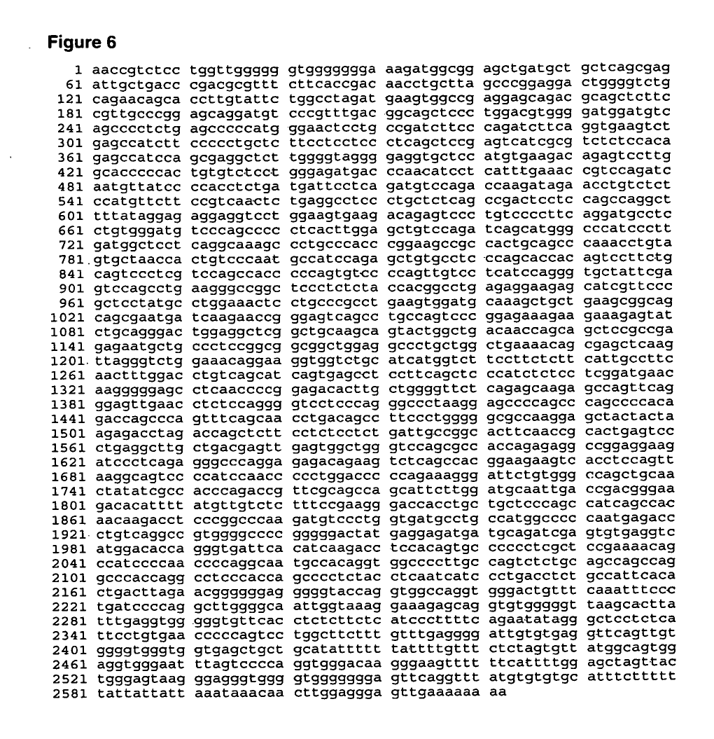

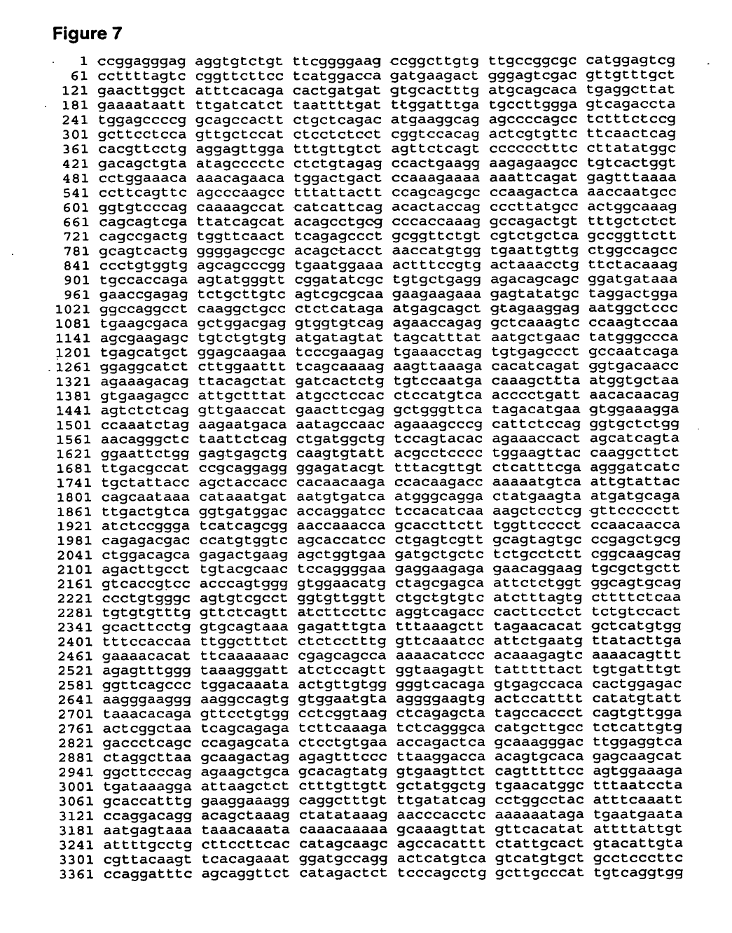

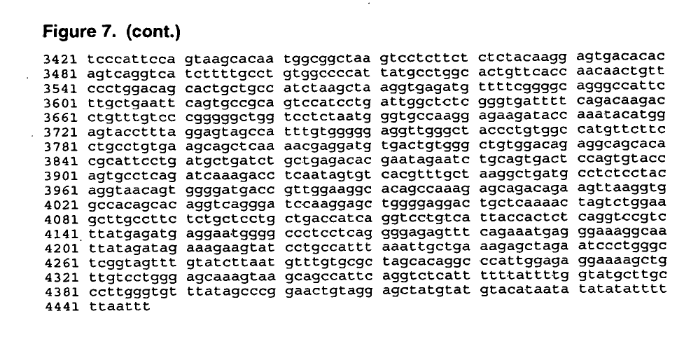

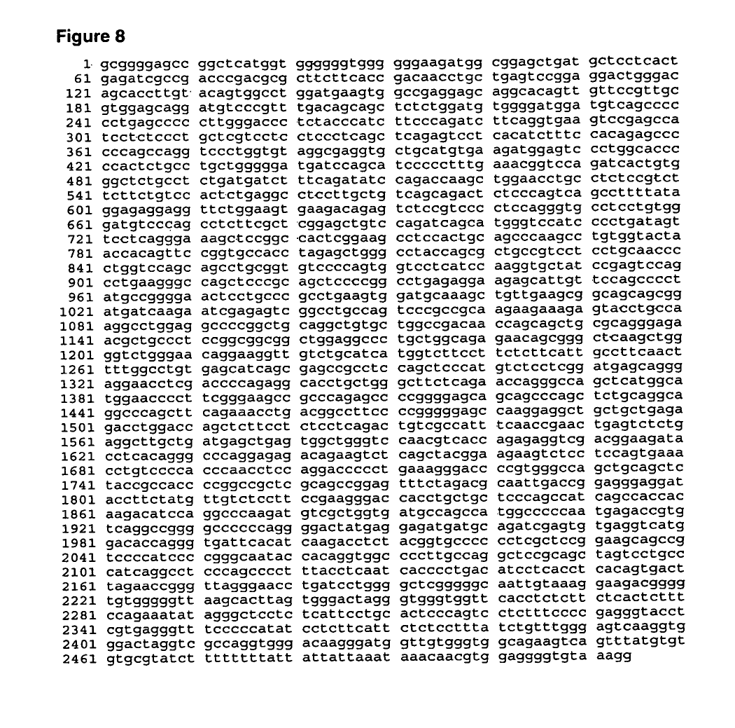

Examples of amino acid and DNA sequences of ATF6 are shown in FIGS. 1-8. Construction of an expression vector comprising ATF6 operably linked to one or more transcriptional regulatory sequences may be accomplished by techniques known to those skilled in the art and the use of appropriate vectors.

An appropriately constructed expression vector may contain, for example, an origin of replication for autonomous replication in the host cells, one or more selectable markers, a limited number of useful restriction enzyme sites, a potential for high copy number, and active promoters. Expression vectors may originate from a variety of sources such as viruses, plasmids, or the cells of a higher organism.

The vectors of the invention may be introduced either ex vivo (where the cells to be transfected are first removed from the subject to be treated) or in vivo, and may result in transient or stable expression on the gene product. Methods of introducing the vectors include viral-based approaches and nonviral approaches, such as lipofection, ligand-DNA conjugates and direct injection of naked DNA. See, e.g., U.S. Pat. No. 6,140,484.

In the case of viral-mediated gene transfer, host cells are transfected with vectors of the present invention by infection with mature virions containing hybrid vectors (the nucleic acid sequences of the invention along with selected viral sequences). The virions used to transfect host cells are preferably replication-defective, such that the virus is not able to replicate in the host cells.

The virions may be produced by co-infection of cultured host cells with a helper virus. Following coinfection, the virions are isolated (e.g., by cesium chloride centrifugation) and any remaining helper virus is inactivated (e.g., by heating). The resulting mature virions contain a vector of the present invention and may be used to infect host cells in the absence of helper virus. Alternatively, high titers of replication-defective recombinant virus, free of helper virus, may be produced in packaging cell lines containing those components for which the virus is defective. Miller, A. D., Hum. Gene Ther. 1:5 (1990).

Several types of viruses, including retroviruses, adeno-associated virus (AAV), herpes virus, vaccinia virus, and several RNA viruses may be amenable for use as vectors in the present invention. Each type of virus has specific advantages and disadvantages, which are appreciated by those of skill in the art. For example, retroviral and AAV vectors are more suitable for stable transfection than are adenovirus, vaccinia virus, or polio virus vectors. Methods for manipulating viral vectors are also known in the art. See, e.g., U.S. Pat. No. 6,140,484.

Retroviruses, like adeno-associated viruses, stably integrate their DNA into the chromosomal DNA of the target cell. Unlike adeno-associated viruses, however, retroviruses typically require replication of the target cells in order for proviral integration to occur. Accordingly, successful gene transfer with retroviral vectors depends on the ability to at least transiently induce proliferation of the target cells.

Retroviral vectors are attractive in part due to the efficiency of transfection-some vectors can stably transduce close to 100% of target cells. The use of retroviral vectors for in vivo gene therapy has been limited, in part, by the requirement of appropriate viral receptors on the target cell. Because the identities of most retroviral receptors are unknown, it has not been possible to determine the distribution of receptors in different cell types. Accordingly, the targeting of specific cell types by retroviral vectors has in many cases proven problematic.

However, this difficulty may be circumvented by modifying the envelope protein of the retrovirus to contain a ligand for a known endogenous (not necessarily viral) receptor expressed on the target cells. An application of this technique is described in detail by Kasahara, N. et al., Science 266:1373 (1994). The virus may also contain an unmodified envelope protein to facilitate cell entry. Adeno-associated viruses are capable of efficiently infecting nondividing cells and expressing large amounts of gene product. Furthermore, the virus particle is relatively stable and amenable to purification and concentration. Replication-defective adenoviruses lacking portions of the E1 region of the viral genome may be propagated by growth in cells engineered to express the E1 genes. Most of the currently-used adenovirus vectors carry deletions in the E1A-E1B and E3 regions of the viral genome. A number of preclinical studies using adenoviral vectors have demonstrated that the vectors are efficient at transforming significant fractions of cells in vivo, and that vector-mediated gene expression can persist for significant periods of time (Rosenfeld, M. A., et al., Cell, 68:143-155 (1992); Quantin, B., et al., Proc. Natl. Acad. Sci. U.S.A. 89:2581 (1992); Stratford-Perricaudet, L. D., et al., J. Clin. Invest. 90:626 (1992); Stratford-Perricaudet, L. D., et al., Bone Marrow Transplant 9(suppl. 1):151 (1992); Jaffe, H. A., et al., Nat. Genet. 1:374 (1992).

Herpes virus vectors are well suited for the delivery and expression of foreign DNA in cells of the central nervous system (CNS), since they can efficiently infect mature, postmitotic neurons. Methods for manipulating the vectors and transfecting CNS cells are well known in the art. See, e.g., U.S. Pat. No. 6,140,484 for various references. Studies utilizing direct injection of vectors into CNS tissue have also been performed (e.g., Zhang, et al., Neuroreport, 3:700 (1992).

In general, the expression vector may be introduced into a host cell via any one of a number of techniques including, but not limited to, transformation, transfection, infection, protoplast fusion, and electroporation. These methods may be used as required in in-vitro or in-vivo procedures.

Plasmids or vectors of the present invention may also be purified and injected directly into a target tissue, using naked DNA injection. Further, liposomes may be employed to deliver genes to target tissues using methods known in the art. See, e.g., U.S. Pat. Nos. 5,208,036, 5,264,618, 5,279,833 and 5,283,185. The liposomes may be constructed to contain a targeting moiety or ligand, such as an antigen, an antibody, or a virus on their surface to facilitate delivery to the appropriate tissue. The liposomes may also be surface-coated, e.g., by incorporation of phospholipid-polyethyleneglycol conjugates, to extend blood circulation time and allow for greater targeting via the bloodstream.

Receptor-mediated endocytic pathways for the uptake of DNA may permit the targeted delivery of genes to specific cell types in vivo. Receptor-mediated methods of gene transfer involve the generation of complexes between plasmid DNA and specific polypeptide ligands (Wu, G. Y., J. Biol. Chem. 266:14338 (1991) that can be recognized by receptors on the cell surface. One of the problems with receptor-mediated uptake for gene delivery is that the endocytic vesicles formed during this process may be transported to the lysosome, where the contents of the endosome are degraded. Methods have been developed to facilitate escape of the DNA from the endosome during the course of its transport. For example, either whole adenovirus (Wagner, E. et al., Proc. Natl. Acad. Sci. U.S.A. 89:6099 (1992) or fusogenic peptides of the influenza HA gene product (Wagner, E., et al., Proc. Natl. Acad. Sci. U.S.A. 89:7934 (1992) may be used to induce efficient disruption of DNA-containing endosomes.

In cases such as those outlined above, where a vector may be targeted to selectively transfect a specific population of cells, it will be understood that in addition to local administration (such as may be achieved by injection into the target tissue), the vector may be administered systemically (e.g., intravenously) in a biologically-compatible solution or pharmaceutically acceptable delivery vehicle. Vector constructs administered in this way may selectively infect the target tissue or may be designed to be selectively expressed in a target tissue (e.g., by way of a tissue-specific promoter).

A variety of mammalian expression vectors may be used to express ATF6 in mammalian cells. Commercially available mammalian expression vectors which may be suitable include, but are not limited to, pMClneo (Stratagene), pXTI (Stratagene), pSG5 (Stratagene), EBOpSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565). Other vectors that have been shown to be suitable expression systems in mammalian cells include the herpes simplex viral based vectors: pHSVI (Geller et al., Proc. Natl. Acad. Sci. USA 87:8950-8954 (1990)); recombinant retroviral vectors: MFG (Jaffee et al., Cancer Res. 53:2221-2226 (1993)); Moloney-based retroviral vectors: LN, LNSX, LNCX, LXSN (Miller and Rosman Biotechniques 7:980-989 (1989)); vaccinia virus vectors: MVA (Sutter and Moss Proc. Natl. Acad. Sci. 89:10847-10851 (1992)); recombinant adenovirus vectors: pJM17 (Ali et al., Gene Therapy 1:367-384 (1994)), (Berkner K. L. Blotechniques 6:616-624 (1988)); second generation adenovirus vectors: DEI/DE4 adenoviral vectors (Wang and Finer, Nature Medicine 2:714-716 (1996)); and adeno-associated viral vectors: AAV/Neo (Muro-Cacho et al., J. Immunotherapy 11:231-237 (1992)). Other gene therapy vectors are disclosed, for example, by Teiger et al., Biomed. Pharmacother. 55:148-54 (2001), and by Buchschacher et al., Hum Gene Ther. 12:1013-19 (2001). Preparation of custom vectors based on any appropriate vector known in the art is also within the scope of the present invention.

Delivery of expression vectors to brain tissue has been described in several publications. For example, U.S. Pat. Nos. 4,866,042, 5,082,670, and 5,529,774, herein incorporated by reference, disclose the use of grafts or implants as one mechanism for introducing retroviral vectors bearing therapeutic gene sequences into the brain. These patents also describe an approach in which the vectors are transmitted across the blood brain barrier. Examples of viral vectors that can be used in the delivery of nucleic acids to the brain according to the invention include herpes simplex virus (HSV) vectors, adenovirus vectors, adeno-associated virus (AAV) vectors, and lentivirus vectors. As an example, WO 98/46273 describes gene delivery using an adeno-associated viral vector that is administered to the brain using a syringe or a catheter. WO 98/46273 also discloses intraventricular delivery in order to obtain widespread, global delivery throughout the brain.

Use of agents that enhance nucleic acid delivery to the brain is also contemplated as part of the invention. For example, intraparenchymal penetration may be enhanced by lowering brain interstitial pressure using systemic mannitol. See, e.g., PCT Patent Application No. WO 98/46273.

Alternate approaches to brain delivery of nucleic acids include injection of naked plasmid DNA as well as liposome-nucleic acid complexes. Suitable lipids and related analogs are described by U.S. Pat. Nos. 5,208,036, 5,264,618, 5,279,833 and 5,283,185. Vectors and DNA encoding an agent can also be adsorbed to or associated with particulate carriers, examples of which include polymethyl methacrylate polymers and polylactides and poly(lactide-coglycolides). See, e.g., McGee et al., J. Micro Encap. (1996).

The nucleic acid vectors of the invention described above may also comprise transcription regulatory sequences such as promoters, enhancers, activators, repressors, terminators, and the like. A promoter may be such that, for example, ATF6 is constitutively expressed or, alternatively, one in which expression is inducible. That is, the promoter may respond to a cellular signal that is always present or to a signal that is only present under certain environmental conditions. As mentioned earlier, examples of promoters that cause expression in the brain include the platlet-derived growth factor (PDGF) promoter (WO 02/26936); prion promoter (Hsiao et al., Science 274:99-102 (1996); and the neuron-specific enolase promoter (Xu R., et al., Gene Ther. 8(17):1323-32 (2001). Examples of promoter/enhancers systems used for the expression of genes in neuronal cells include the promoter/enhancer from the immediate early (1E) human cytomegalovirus (hCMV) (WO 98/46273 and WO 99/50404), and the promoter/enhancer from the human neurofilament-light (hNF-L) gene (Vidal-Sanz M., et al., Eur J Neurosci. 3(8):758-763 (1991). Examples of terminators used in mammalian systems include those from the cytomegalovirus and SV40 systems, as well as the bovine growth hormone (BGH) polyadenylation sequence. See, e.g., WO 98/55616; WO 02/16594 and U.S. Pat. Nos. 6,248,555 and 6,323,030.

Functional Forms of ATF6

ATF6 is a transmembrane transcription factor having a cytosolic N-terminal domain (about 370-380 amino acids long), a 21-residue transmembrane domain, and a 270-residue extracytoplasmic domain that projects into the ER lumen. Upon activation, for example, in response to the accumulation of unfolded proteins in the ER, ATF6 is cleaved to release the N-terminal domain.

Therefore, a nucleic acid sequence encoding a functional form of ATF6 may encode the full-length form of ATF6, the N-terminal domain of ATF6, and/or a functional derivative of either the full-length or the N-terminal domain of ATF6.

Site-1 protease (S1P) and site-2 protease (S2P) are involved in the cleavage of ATF6 to release the N-terminal domain into the cytosol. Ye at al (2000) discloses the location of amino acids where cleavage of ATF6 may occur, including the proposed location of the transmembrane domain. Therefore, the N-terminal domain of ATF6 may include polypeptide chains that start at amino acid no. 1 and end at any of the amino acids that are part of the transmembrane domain. That is, the N-terminal domain of ATF6 may be a polypeptide chain starting at amino acid no. 1 and ending at any residue from amino acid no. 378 to amino acid no. 398. For example, the N-terminal domain of ATF6 may end at those amino acids where enzymatic cleavage by S1P or S2P occurs. A nucleic acid sequence encoding the desired N-terminus amino acid sequence of ATF6 may be prepared by recombinant techniques known in the art.

Additionally, a nucleotide sequence comprising the bZIP domain of ATF6 is also considered a functional form of ATF6. This statement is based on the discovery by Wang et al. that a peptide comprising the bZIP domain of ATF6 was able to bind an ATF6 consensus DNA sequence and presumably activate transcription of UPR-regulated genes. Wang et al., J. Biol. Chem. 275:27013-27020 (2000).

Derivatives of either the full-length ATF6, the N-terminal domain of ATF6, or a peptide comprising the bZIP domain of ATF6 (hereinafter derivatives of ATF6) may be produced by techniques known in the art, including, but not limited to, deletions, additions, conservative amino acid substitutions, or other manipulations that produce a molecule that maintains the property of preventing of cell-death when overexpressed.

Ample evidence demonstrates that derivatives of natural proteins often retain a biological activity similar to that of the naturally occurring protein. For example, Gayle and coworkers conducted extensive mutational analysis of human cytokine IL-Ia. Gayle et al. J. Biol. Chem. 268:22105-22111 (1993). They used random mutagenesis to generate over 3,500 individual IL-Ia mutants that averaged 2.5 amino acid changes per variant over the entire length of the molecule. Multiple mutations were examined at every possible amino acid position. The investigators found that “[m]ost of the molecule could be altered with little effect on either (binding or biological activity].” In fact, only, 23 unique amino acid sequences, out of more than 3,500 nucleotide sequences examined, produced a protein that significantly differed in activity from that of the wild-type.

Guidance concerning how to make phenotypically silent amino acid substitutions is provided in Bowie et al., “Deciphering the Message in Protein Sequences: Tolerance to Amino Acid Substitutions,” Science 247:1306-1310 (1990). The authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection are likely positions that are not critical for protein function. Thus, positions that tolerate amino acid substitution could be modified while still maintaining biological activity of the protein.

The second strategy uses genetic engineering to introduce amino acid changes at specific positions of a cloned gene to identify regions critical for protein function. For example, site directed mutagenesis by alanine-scanning mutagenesis (introduction of single alanine mutations at every residue in the molecule) may be used. Cunningham and Wells, Science 244:1081-1085 (1989).

Bowie et al., Science 247:1306-1310 (1990), indicate that these two strategies have shown that proteins are surprisingly tolerant of amino acid substitutions. The authors further dsclose which amino acid changes are likely to be permissive at certain amino acid positions in the protein. For example, most buried (within the tertiary structure of the protein) amino acid residues require nonpolar side chains, whereas few features of surface side chains are generally conserved.

In general, amino acid substitutions made in order to prepare derivatives of ATF6 are accomplished by selecting substitutions that do not differ significantly in their effect on maintaining, for example, (a) the structure of the ATF6 peptide backbone in the area of the substitution, (b) the charge or hydrophobicity of ATF6 at the target site, or (c) the bulk of a side chain. Naturally occurring residues are divided into groups based on common side-chain properties, for example:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic; trp, tyr, phe.

Therefore, conservative amino acid substitutions include, for example, substitution of an aspartic acid residue by a glutamic acid residue because both are acidic amino acids. Similarly, the following examples show acceptable conservative substitutions: lysine/arginine/histidine as basic amino acids; leucine/isoleucine, methionine/valine, alanine/valine as hydrophobic amino acids; serine/glycine/alanine/threonine as hydrophilic amino acids.

Conservative amino acid substitutions also include groupings based on side chains. For example, substitutions include substitutions among amino acids that belong to a group having aliphatic side chains, such as glycine, alanine, valine, leucine, and isoleucine. Similarly, substitutions among a group of amino acids having aliphatic-hydroxyl side chains include substitutions between serine and threonine; substitutions among a group of amino acids of amide-containing side chains include substitutions between asparagine and glutamine; substitutions among a group of amino acids having aromatic side chains include substitutions between phenylalanine, tyrosine, and tryptophan; substitutions among a group of amino acids having basic side chains include substitutions between lysine, arginine, and histidine; and substitutions among a group of amino acids having sulfur-containing side chains include substitutions between cysteine and methionine.

Therefore, one skilled in the art can predict based on these guidelines that replacement of a leucine with an isoleucine or valine, an aspartate with a glutamate, a threonine with a serine, or a similar replacement of an amino acid with a structurally related amino acid will not have a major effect on the properties of the resulting ATF6 derivative.

Additionally, computer programs that predict the effect of a specific amino acid substitution or mutation on the structure of the protein are available. PCT Patent Application No. WO 02/543063 discloses one such method and also reviews the state of the art in this field.

Whether any amino acid change (deletion, addition, substitution, or a combination thereof) results in a functional ATF6 derivative can readily be determined by assaying the cell-death prevention activity of the derivative.

In addition to conservative amino acid substitutions, ATF6 derivatives used in the instant invention include (i) substitutions with one or more non-conserved amino acid residues, where the substituted amino acid residue may be a chemically modified amino acid (e.g., by methylation, acylation, etc.) that may or may not be encoded by the genetic code, (ii) substitutions with one or more amino acid residues having a substituent group, (iii) fusion of AFT6 with another compound, such as a compound to increase the stability and/or solubility of ATF6 (for example, polyethylene glycol), or (iv) fusion of the AFT6 polypeptide with additional amino acids. Examples of preparation of derivatives following these guidelines can be found in, for example, U.S. Pat. No. 5,876,969, EP Patent 0 413 622, and U.S. Pat. No. 5,766,883. Such derivatives of ATF6 may be administered directly to a cell or subject instead of administering a nucleic acid encoding for a derivative of ATF6.

Derivatives of ATF6 that contain deletions or additions of amino acid residues may be produced by using known methods of protein engineering and recombinant DNA technology. For instance, one or more amino acids can be deleted or added from either the N-terminus or C-terminus of ATF6 (or both terminus) without substantial loss of biological function.

For example, Ron and coworkers reported variant keratinocyte growth factor (KGF) proteins having heparin binding activity even after deleting 3, 8, or 27 amino-terminal amino acid residues. Ron et al. J. Biol. Chem. 268:2984-2988 (1993). Similarly, interferon gamma exhibited up to ten times higher activity after deleting 8-10 amino acid residues from the carboxy terminus of the protein. Dobeli et al., J. Biotechnol. 7:199-216 (1988).

Additionally, derivatives of ATF6 may be prepared by modification of the amino acids in ATF6 or in a derivative of ATF6. Modifications may occur anywhere in ATF6 or its derivative, including the peptide backbone, the amino acid side-chains and the amino or carboxyl termini. Modifications may include, for example, acetylation, acylation, ADP-ribosylation, amidation, covalent attachment of other functional moiety, covalent attachment of a lipid or lipid derivative, covalent attachment of phosphotidylinositol, cross-linking, cyclization, disulfide bond formation, demethylation, formation of covalent cross-links, formylation, gamma-carboxylation, glycosylation, glycophosphatidylinositol (GPI) anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, E. Creighton Proteins—Structure and Molecular Properties, 2nd Ed., W. H. Freeman and Company, New York (1993); B. C. Johnson, Post Translational Covalent Modification of Proteins, Academic Press, New York, (1983); Seifter et al., Meth. Enzymol. 182:626-646 (1990); Rattan et al., Ann. N.Y. Acad. Sci. 663:48-62 (1992). Preparation of these modified derivatives may, for example, be useful if direct administration of the derivative, rather than administration of a nucleic acid encoding the derivative, is contemplated.

Because of the degeneracy of genetic code, the skilled artisan may prepare more than one nucleic acid sequence that encodes ATF6 or one of its derivatives. Administration of nucleic acids obtained in this manner is also encompassed by the instant invention.

Use of natural and non-natural allelic variations of ATF6 is also contemplated as being part of the invention. These allelic variants can vary at either the polynucleotide and/or polypeptide level. Non-naturally occurring variants may be produced by mutagenesis techniques or by direct synthesis. Therefore, the present invention is also directed to proteins containing polypeptides at least 80%, 85%, 90%, 95%, 96%, 97%, 98% or 99% identical to the ATF6 polypeptide sequences shown in FIGS. 1-4. The methods of the invention may also utilize nucleic acid molecules comprising, or alternatively, consisting of, a polynucleotide sequence at least 70%, 75%, 80%, 85%, 90%, 95%, 96%, 97%, 98% or 99% identical to the polynucleotide sequence encoding ATF6. Whether any particular nucleic acid molecule or polypeptide is at least 80%, 85%, 90%, 95%, 96%, 97%, 98% or 99% identical to an ATF6 sequence can be determined conventionally using known computer programs. See, e.g., WO 01/54474.

Nucleic acids encoding these homologous polynucleotide sequences can be isolated by hybridization under different conditions. It is well known in the art that the degree of stringency (high, intermediate, low, etc.) depends on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature of the probe to about 20° C. to 25° C. below the melting temperature). Functionally, maximum stringency conditions may be used to identify nucleic acid sequences having strict identity or near-strict identity with the hybridization probe; while high stringency conditions may be used to identify nucleic acid sequences having about 80% or more sequence identity with the probe. Persons skilled in the art will appreciate that one or more factors be may be varied to generate conditions of stringency that may be different from each other in temperature and ionic strength composition but that are equivalent to in terms of the results desired. One general guide for nucleic acid hybridization is Tijssen, “Laboratory Techniques in Biochemistry and Molecular Biology” Elsevier, vol. 24, Hybridization with Nucleic Acid Probes, Part I Overview of principles of hybridization and the strategy of nucleic acid assays (1993).

Whether any homologous polynucleotide sequence encodes a functional ATF6 derivative can readily be determined by assaying the cell-death prevention activity of the derivative.

Inducing Expression of UPR-Regulated Genes

As mentioned previously, ATF6 acts as a transcription factor and binds to the ERSE, thereby inducing the expression of UPR-responsive genes. As expected, however, other basal transcription factors are required before transcription of these genes can occur. Some of these basal transcription factors include NF-Y and YY1. van Laar et al., Current Protein and Peptide Science 2:169-190 (2001).

Therefore, in one embodiment of the present invention the method for preventing cell death may comprise introducing a vector comprising a nucleic acid sequence encoding a basal transcription factor that binds to the ERSE. For example, the vector may comprise a nucleic acid sequence encoding NF-Y, YY1, or both. Furthermore, the vector may optionally comprise a nucleic acid sequence encoding ATF6.

Furthermore, a method for preventing cell death may comprise any known method in the art that induces the expression of endogenous ATF6. For example, a method for inducing the expression of ATF6 may include inducing the UPR. This induction may take place by the accumulation of unfolded proteins in the ER or by inducing the execution of any intermediate step in the signaling pathway between the accumulation of unfolded proteins in the ER and the expression of ATF6.

In mammalian cells, one such intermediate step includes the activation of the transmembrane kinase Ire1, of which two isoforms have been identified (activation of Ire1 is induced by the accumulation of unfolded proteins in the ER). van Laar et al., Current Protein and Peptide Science 2:169-190 (2001). Activation of Ire1 involves dimerization and autophosphorylation, after which presenilin 1 and 2 cleave the kinase-nuclease domain of Ire1. This cleavage occurs in a RIP-like fashion analogous to the release of the N-terminus of ATF6 after cleavage by SP1 and/or SP2.

Although ATF6 may be activated by an Ire1-independent mechanism induced only by accumulation of unfolded proteins in the ER, active ATF6 may also be produced by an Ire1-catalyzed splicing of ATF6 mRNA that results in a fragment encoding only the soluble domain of ATF6. van Laar et al., Current Protein and Peptide Science 2:169-190 (2001), hereby incorporated by reference in its entirety.

Therefore, a method for inducing the expression of ATF6 may comprise, for example, introducing into a cell a vector comprising a nucleic acid sequence encoding one of more of the following molecules: any one of the transmembrane isoforms of Ire1, any soluble form of Ire1, presenilin 1, and/or presenilin 2.

Additionally, a method for inducing the expression of ATF6 may also comprise introducing into a cell a vector comprising a nucleic acid sequence encoding one of more transcription factors for any of the following genes: any one of the transmembrane isoforms of Ire1, any soluble form of Ire1, presenilin 1, presenilin 2 and ATF6.

Furthermore, because processing of ATF6 occurs via proteolysis by S1P and/or S2P, another method for inducing the expression of ATF6 may comprise introducing into a cell a vector comprising a nucleic acid sequence encoding S1P, S2P, or both, wherein this vector optionally comprises a nucleic acid sequence encoding ATF6.

Alternatively, induction of ATF6 may also be effected by exposing the host cell to suitable environmental conditions. An example of these conditions include incubation of cell in the presence of tunicamycin, or in the presence of any other inhibitor of protein folding known in the art.

Homologous and Nonhomologous Recombination

Because the original endogenous promoter activates transcription of the ATF6 gene only under conditions that trigger the UPR, it may be advantageous to replace this promoter with a promoter that allows constitutive expression of the ATF6 gene or at least enhanced expression with respect to the endogenous expression. Therefore, in one embodiment of the invention the promoter sequence of ATF6 is targeted for replacement by a nucleic acid sequence comprising an exogenous promoter sequence (not the original promoter).

The nucleic acid sequence to be inserted by homologous recombination may comprise a promoter flanked by two targeting regions of sufficient length. These targeting regions comprise nucleotide sequences flanking the endogenous promoter of ATF6. A sufficient length for each targeting region sequence is normally between 50-200 bp, although the skilled artisan will recognize that other lengths may also be used successfully. The exogenous promoter may be, for example, the human cytomegalovirus (CMV) enhancer/promoter.

In addition to using sequences unique to the target region, the skilled artisan may also use sequences that are homologous to repetitive sequences found in the mammalian genome, such as the Alu, LINE, THE, etc., sequences. See, e.g., U.S. Pat. No. 6,015,708. These sequences may be present one or multiple times on the targeting region sequence flanking the nucleic acid sequence to be inserted by homologous recombination.

In another embodiment of the invention, the endogenous gene encoding ATF6 and the corresponding promoter in a cell are replaced by a nucleic acid sequence encoding one or more copies of an ATF6 gene (e.g., either the full length or soluble domains) placed under the control of a exogenous promoter, for example the human cytomegalovirus (CMV) enhancer/promoter. Optionally, the nucleic acid sequence may also comprise sequences coding for S1P, S2P, or both. As mentioned previously in a similar context, these nucleic acid sequences may contain additional transcriptional regulatory sequences that modify the expression of the gene of interest such as, for example, by increasing the rate of transcription, increasing translation efficiency, increasing mRNA accumulation, making the expression inducible, etc.

The nucleic acid sequence to be inserted by homologous recombination may optionally include at least one selectable marker gene, such as a gene providing a particular antibiotic resistance. Furthermore, the nucleic acid sequence to be inserted by homologous recombination may include an amplifiable gene, which may also allow amplification of the gene of interest. Amplifiable genes include dihydrofolate reductase (DHFR, metallothionein-I and -II, for example of primate origin, adenosine deaminase, ornithine decarboxylase, glutamate synthase, etc. In some cases, the selectable marker and the amplifiable marker may be the same.

In another embodiment of the invention, the nucleic acid sequence encoding one or more copies of an ATF6 gene and suitable transcriptional regulatory sequences does not replace the endogenous ATF6 gene, but is inserted at a different region in the genome. In this case, the nucleic acid sequence includes two targeting segments, which, while separated from one another in the sequence by those elements to be inserted into the genome, may be contiguous in the native genome. The skilled artisan will recognize that there is no criticality as to the specific location of the inserted nucleic acid sequence within the genome. It is only important that it be taken up somewhere within the nucleus because both the regulatory segment or segments and the exogenous gene (as well as any selectable markers and amplifiable genes) are inserted together. Homologous recombination is described, for example, in U.S. Pat. Nos. 5,282,071, and 5,578,461.

In another embodiment of the invention, a nucleic acid sequence encoding one or more copies of an ATF6 gene and suitable transcriptional regulatory sequences is introduced into a cell by nonhomologous or nontargeted recombination. That is, the nucleic acid sequences in this embodiment of the invention do not contain target sequences. In most cases, nonhomologous recombination is thought to occur through the joining of free nucleic acid ends that contain an end capable of being joined to a second nucleic acid end, either directly, or following repair or processing. The nucleic acid end may consist of a 5′ overhang, a 3′ overhang, or blunt end. As mentioned previously, these nucleic acid sequences may optionally comprise sequences coding for S1P, S2P, a selectable marker, or an amplifiable marker.

As with homologous recombination of nucleic acid sequences comprising both the regulatory segment or segments and the exogenous gene (as well as any selectable markers and amplifiable genes), the skilled artisan will recognize that there is no criticality as to the specific location of the inserted nucleic acid sequence within the cell. Nonhomologous recombination is described, for example, in PCT Application No. WO 00/49162.

The techniques of homologous and nonhomologous recombination may be used to introduce, into a cell, nucleotide sequences encoding any of the molecules expressly mentioned in this application or any other molecule that would be apparent to the skilled artisan as being useful in the methods of the instant invention upon reading the instant application as a whole.

Although some embodiments of the invention have been described in terms of introducing into a cell a vector comprising a nucleic acid sequence encoding a particular molecule, the methods of the invention also contemplate the introduction of a vector comprising a nucleic acid sequence encoding a functional derivative of that molecule, or any other molecule that would be apparent to the skilled artisan as useful in the methods of the instant invention upon reading the application as a whole.

Additionally, the present invention also contemplates combination in a single embodiment of one or more of the methods described throughout the specification for the purpose of preventing cell death.

Compositions

The present invention also provides compositions useful in carrying out the methods of the invention.

These compositions may contain an agent in ranges from about 0.1 to about 10% of the composition. In another embodiment, these compositions may contain an agent in ranges from about 0.5 to about 5% of the composition. In yet another embodiment, these compositions may contain an agent in ranges from about 1 to about 3% of the composition. An agent in the context of the compositions of the invention is any molecule (nucleic acid sequence, protein, etc.) identified in this application as useful in the methods of the instant invention.

The compositions of the invention may also be used in appropriate association with other pharmaceutically active compounds. The following methods and excipients are merely exemplary and are in no way meant to be limiting.

For oral compositions, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents.

The compositions may include, depending on the composition desired, pharmaceutically acceptable, nontoxic carriers or diluents, which are defined as vehicles commonly used to formulate compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the composition may also include other carriers, adjuvants, or nontoxic, nontherapeutic, nonimmunogenic stabilizers and the like. Also included may be carrier molecules such as proteoglycans. Specific examples of such carrier molecules include, but are not limited to, glycosaminoglycans such as heparin sulfate, hyaluronic acid, keratin-sulfate, chondroitin 4-sulfate, chondroitin 6-sulfate, heparin sulfate and dermatin sulfate, perlecan, and pento polysulfate.

The agents of the invention may be formulated into preparations for injection by dissolving, suspending, or emulsifying the agent in a physiologically acceptable diluent with a carrier. Carriers include sterile liquids, such as water, oils, with or without the addition of a surfactant, and glycols. Oils may be petroleum derivatives, or of animal, vegetable, and synthetic origin, for example, peanut oil, soybean oil, and mineral oil. Examples of glycols include propylene glycol and polyethylene glycol. The compositions may also contain conventional additives such as solubilizers, isotonic agents, suspending agents, emulsifying agents, stabilizers and preservatives. The agents of this invention may also be used in a sustained release form, for example a depot injection, implant preparation, or osmotic pump, which can be formulated in such a manner as to permit a sustained release of the active ingredient.

The agents can be utilized in an aerosol composition to be administered via inhalation or pulmonary delivery. The agents of the present invention may be formulated into pressurized acceptable propellants such as dichlorodifluoromethane, nitrogen, and the like.

Administration of an agent of the invention may be accomplished by any convenient means, including parenteral injection, and may be systemic or localized in delivery. The agents of this invention can be incorporated into a variety of compositions for therapeutic administration. In general, the agents of the present invention can be formulated into compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the agents can be achieved in various ways, including oral, buccal, rectal, parenteral, intraperitoneal, intradermal, transdermal, intratracheal, intrathecal, intranasal, gastric, intramuscular, intracranial, subdermal, etc., administration. The active agent may be systemic after administration or may be localized by the use of regional administration, intramural administration, or use of an implant that acts to retain the active component at the site of implantation.

Unit dosage forms for oral or rectal administration such as syrups, elixirs, and suspensions may be provided wherein each dosage unit, for example, teaspoonful, tablespoonful, tablet, or suppository, contains a predetermined amount of the composition containing one or more agents of the present invention. Similarly, unit dosage forms for injection or intravenous administration may comprise the agent of the present invention in a composition as a solution in sterile water, normal saline, or another pharmaceutically acceptable carrier.

Doses for nucleic acids encoding other agents of the invention range from about 10 ng to about 1 g, from about 100 ng to about 100 mg, from about 1 μg to about 10 mg, or from about 30 to about 300 μg DNA per subject. Doses for infectious viral vectors vary from about 10 to about 100, or more, virions per dose.

Those of skill in the art will readily appreciate that dose levels can vary as a function of the specific agent, the severity of the symptoms, and the susceptibility of the subject to side effects. Additionally, some of the specific agents of the invention may be more potent than others. Dosages for a given agent are readily determinable by those of skill in the art by a variety of means, for example by measuring the relative physiological potency of a given agent by methods known in the art with respect to the potency of another agent and adjusting the dosage accordingly.

Implants for sustained release compositions are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is, for example, placed in proximity to the site of protein deposits (e.g., the site of formation of protein deposits associated with neurodegenerative disorders), so that the local concentration of active agent is increased at that site relative to the rest of the body.

The compositions can also be administered by infusion into the brain, and may be administered in either a continuous (e.g., sustained) or non-continuous fashion. Methods, compositions, and devices suitable for delivery to the brain in a continuous (e.g., chronic) or non-continuous (e.g., single, discrete dose per administration) fashion are described in, for example, U.S. Pat. Nos. 5,711,316; 5,832,932; 5,814,014; 5,782,798; 5,752,515; 5,735,814; 5,713,923; 5,686,416; 5,624,898; 5,624,894; 5,124,146; and 4,866,042.

A typical dosage unit for administration to a subject includes, but is not limited to: a solution suitable for intravenous administration; a tablet taken from two to six times daily; or a time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Screening Method

The present invention also provides for a method to screen for substances that affect ATF6 activity.

In one embodiment, the method can determine whether a substance increases or decreases the activity of at least one functional form of ATF6 in a cell whose production by a cell evokes a responsive change in at least one characteristic. This method can comprise providing two cell lines, one which produces at least one functional form of ATF6 and a second which produces the at least one functional form of ATF6 at a lower level than the first cell line, or does not produce the at least one functional form of ATF6 at all. Then, the substance can be placed in contact with the first and second cell lines. Finally, one can compare the at least one characteristic of the first cell line with the second cell line.

In exemplary embodiments, the first and second cell lines are neurons or nigral neurons.

In exemplary embodiments, the at least one characteristic is chosen from a characteristic that is observable with the naked eye, a characteristic that is a cultural characteristic of the cell, a characteristic that is a morphological characteristic, and combinations thereof. Additionally, the at least one characteristic can be a measure of an amount of ATF6 mRNA, or a measure of an amount of at least one functional form of ATF6 protein.

In other exemplary embodiments, the at least one characteristic can be a rate of cell death, a rate of cell proliferation, or combinations thereof.

In other exemplary embodiments, the at least one characteristic can be a response along the UPR signaling pathway. This response can be an expression of a gene chosen from Ire1, presenilin 1, presenilin 2, ATF6 regulated genes, or combination thereof.

In other exemplary embodiments, the at least one characteristic can be an expression of a UDR-regulated gene. The UDR-regulated gene can be a molecular chaperone BiP gene, a gene involved in protein glycosylation, a gene involved in protein secretion, a gene involved in protein degradation, a gene involved in membrane biosynthesis, or combinations thereof.

In other exemplary embodiments, the characteristic can be a cleavage rate of a full length ATF6, a rate of protein folding, a measure of an amount of protein accumulation, a measure of an amount of protein aggregation, a measure of an amount of protein precipitation, a measure of an amount of protein plaques, or combinations thereof.

Additionally, the present invention also contemplates other methods of screening which are combinations of those mentioned above, or are implied in the previously-described other embodiments of the invention.

Please incorporate the paper copy of the sequence listing at the end of the specification.

We claim:

1. A method for preventing cell death in a subject in need thereof comprising, administering to the subject an effective amount of a composition comprising a nucleic acid encoding at least one functional form of ATF6.

2. The method according to claim 1, whereby the at least one functional form of ATF6 is chosen from a full-length form of ATF6, an N-terminal domain form of ATF6, a bZIP-ATF6 fragment, functional derivatives thereof, and combinations thereof.

3. The method according to claim 2, whereby the full-length form of ATF6 is chosen from human ATF6-α, human ATF6-β, murine ATF6-α, and murine ATF6-β.

4. The method according to claim 1, whereby the at least one functional form of ATF6 is a full-length form of ATF6 or an N-terminal domain form of ATF6 having at least one modification chosen from deletions, additions, amino acid substitutions, conservative amino acid substitutions, and combinations thereof.

5. The method according to claim 1, whereby the nucleic acid further comprises a nucleic acid sequence encoding at least one basal transcription factor that binds to the ER-stress responsive element.

6. The method according to claim 5, whereby the at least one basal transcription factor is chosen from NF-Y, YY1, functional derivatives thereof, and combinations thereof.

7. The method according to claim 1, whereby the nucleic acid further comprises at least one transcriptional regulatory sequence.

8. The method according to claim 7, whereby the at least one transcriptional regulatory sequence is chosen from promoters, enhancers, activators, terminators, polyadenylation signals, and combinations thereof.

9. The method according to claim 1, whereby the nucleic acid further comprises a nucleic acid sequence that induces expression of a functional form of ATF6.

10. The method according to claim 9, whereby the nucleic acid sequence that induces expression of a functional form of ATF6 is chosen from Ire1, presenilin 1, preselinin 2, transcription factors of ATF6, site-1 protease, site-2 protease, promoters that replace the original promoter of the gene encoding ATF6 in the cell's genome, enhancers that replace the original promoter of the gene encoding ATF6 in the cell's genome, and combinations thereof.

11. The method according to claim 1, whereby the administering is used as a stand-alone therapy.

12. The method according to claim 1, whereby the nucleic acid further comprises a vector chosen from viral vectors, plasmids, and cosmids.

13. The method according to claim 12, whereby the vector is a mammalian vector.

14. The method according to claim 12, whereby the vector directs nucleic acid delivery to the brain.

15. The method according to claim 12, whereby the vector is chosen from neural-specific expression vectors, blood brain barrier transmission-specific vectors, vectors comprising a neural-specific promoter, and combinations thereof.

16. The method according to claim 12, whereby the vector is chosen from retroviruses, adeno-associated viruses, herpes viruses, vaccinia viruses, RNA viruses, herpes simplex virus vectors, adenovirus vectors, adeno-associated virus vectors, lentivirus vectors, vectors comprising a platlet-derived growth factor promoter, vectors comprising a prion promoter, vectors comprising a neuron-specific enolase promoter, vectors comprising promoter/enhancer systems from an immediate early human cytomegalovirus, vectors comprising promoter/enhancer systems from a human neurofilament-light gene, vectors comprising terminators from a cytomegalovirus system, vectors comprising terminators from an SV40 system, and vectors comprising terminators from a bovine growth hormone polyadenylation sequence.

17. The method according to claim 1, whereby the composition further comprises at least one agent that enhances nucleic acid delivery to the brain.

18. The method according to claim 17, whereby the at least one agent that enhances nucleic acid delivery to the brain is mannitol.

19. The method according to claim 1, whereby the composition further comprises at least one pharmaceutically acceptable additive, lubricant, diluent, buffer, moistening agent, preservative agent, flavoring, adjuvant, carrier, stabilizer, suspending agent, emulsifying agent, propellant, or other vehicle.

20. A method for preventing cell death in a cell in need thereof comprising, administering to the cell an effective amount of a composition comprising a nucleic acid encoding at least one functional form of ATF6.

21. The method according to claim 20, whereby the at least one functional form of ATF6 is chosen from a full-length form of ATF6, an N-terminal domain form of ATF6, a bZIP-ATF6 fragment, functional derivatives thereof, and combinations thereof.

22. The method according to claim 21, whereby the full-length form of ATF6 is chosen from human ATF6-α, human ATF6-β, murine ATF6-α, and murine ATF6-β.

23. The method according to claim 20, whereby the at least one functional form of ATF6 is a full-length form of ATF6 or an N-terminal domain form of ATF6 having at least one modification chosen from deletions, additions, amino acid substitutions, conservative amino acid substitutions, and combinations thereof.

24. The method according to claim 20, whereby the nucleic acid further comprises a nucleic acid sequence encoding at least one basal transcription factor that binds to the ER-stress responsive element.

25. The method according to claim 24, whereby the at least one basal transcription factor is chosen from NF-Y, YY1, functional derivatives thereof, and combinations thereof.

26. The method according to claim 20, whereby the nucleic acid further comprises at least one transcriptional regulatory sequence.

27. The method according to claim 26, whereby the at least one transcriptional regulatory sequence is chosen from promoters, enhancers, activators, terminators, polyadenylation signals, and combinations thereof.

28. The method according to claim 20, whereby the nucleic acid further comprises a nucleic acid sequence that induces expression of a functional form of ATF6.

29. The method according to claim 28, whereby the nucleic acid sequence that induces expression of a functional form of ATF6 is chosen from Ire1, presenilin 1, preselinin 2, transcription factors of ATF6, site-1 protease, site-2 protease, promoters that replace the original promoter of the gene encoding ATF6 in the cell's genome, enhancers that replace the original promoter of the gene encoding ATF6 in the cell's genome, and combinations thereof.

30. The method according to claim 20, whereby the nucleic acid further comprises a vector chosen from viral vectors, plasmids, and cosmids.

31. The method according to claim 30, whereby the vector is a mammalian vector.

32. The method according to claim 30, whereby the vector is chosen from neural-specific expression vectors, blood brain barrier transmission-specific vectors, vectors comprising a neural-specific promoter, and combinations thereof.

33. The method according to claim 30, whereby the vector is chosen from retroviruses, adeno-associated viruses, herpes viruses, vaccinia viruses, RNA viruses, herpes simplex virus vectors, adenovirus vectors, adeno-associated virus vectors, lentivirus vectors, vectors comprising a platlet-derived growth factor promoter, vectors comprising a prion promoter, vectors comprising a neuron-specific enolase promoter, vectors comprising promoter/enhancer systems from an immediate early human cytomegalovirus, vectors comprising promoter/enhancer systems from a human neurofilament-light gene, vectors comprising terminators from a cytomegalovirus system, vectors comprising terminators from an SV40 system, and vectors comprising terminators from a bovine growth hormone polyadenylation sequence.

34. The method according to claim 20, whereby the composition further comprises at least one pharmaceutically acceptable additive, lubricant, diluent, buffer, moistening agent, preservative agent, flavoring, adjuvant, carrier, stabilizer, suspending agent, emulsifying agent, propellant, or other vehicle.

35. A method for preventing cell death in a subject in need thereof comprising, administering to the subject an effective amount of a composition comprising at least one functional form of ATF6.

36. The method according to claim 35, whereby the at least one functional form of ATF6 is chosen from a full-length form of ATF6, an N-terminal domain form of ATF6, a bZIP-ATF6 fragment functional, derivatives thereof, and combinations thereof.

37. The method according to claim 36, whereby the full-length functional form is chosen from human ATF6-α, human ATF6-β, murine ATF6-α, and murine ATF6-β.

38. The method according to claim 35, whereby the at least one functional form of ATF6 is a full-length form of ATF6 or an N-terminal domain form of ATF6 having at least one modification chosen from deletions, additions, amino acid substitutions, conservative amino acid substitutions, and combinations thereof.

39. The method according to claim 35, whereby the at least one functional form of ATF6 comprises at least one covalent modification.

40. The method according to claim 39, whereby the at least one modification occurs in at least one region chosen from the peptide backbone, the amino acid side-chains, the amino terminus and the carboxyl terminus.

41. The method according to claim 39, whereby the at least one modification is chosen from acetylation, acylation, ADP-ribosylation, amidation, covalent attachment of a functional moiety, covalent attachment of a lipid, covalent attachment of a lipid derivative, covalent attachment of phosphotidylinositol, cross-linking, cyclization, disulfide bond formation, demethylation, formation of covalent cross-links, formylation, gamma-carboxylation, glycosylation, glycophosphatidylinositol anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins, arginylation, and ubiquitination.

42. The method according to claim 35, whereby the administering is used as a stand-alone therapy.

43. The method according to claim 35, whereby the composition further comprises at least one pharmaceutically acceptable additive, lubricant, diluent, buffer, moistening agent, preservative agent, flavoring, adjuvant, carrier, stabilizer, suspending agent, emulsifying agent, propellant, or other vehicle.

44. A method for preventing cell death in a cell in need thereof comprising, administering to the cell an effective amount of a composition comprising at least one functional form of ATF6.

45. The method according to claim 44, whereby the at least one functional form of ATF6 is chosen from a full-length form of ATF6, an N-terminal domain form of ATF6, a bZIP-ATF6 fragment, functional derivatives thereof, and combinations thereof.

46. The method according to claim 45, whereby the full-length functional form is chosen from human ATF6-α, human ATF6-β, murine ATF6-α, and murine ATF6-β.

47. The method according to claim 44, whereby the at least one functional form of ATF6 is a full-length form of ATF6 or an N-terminal domain form of ATF6 having at least one modification chosen from deletions, additions, amino acid substitutions, conservative amino acid substitutions, and combinations thereof.

48. The method according to claim 44, whereby the at least one functional form of ATF6 comprises at least one covalent modification.

49. The method according to claim 48, whereby the at least one modification occurs in at least one region chosen from the peptide backbone, the amino acid side-chains, the amino terminus and the carboxyl terminus.

50. The method according to claim 48, whereby the at least one modification is a from acetylation, acylation, ADP-ribosylation, amidation, covalent attachment of other functional moiety, covalent attachment of a lipid, covalent attachment of a lipid derivative, covalent attachment of phosphotidylinositol, cross-linking, cyclization, disulfide bond formation, demethylation, formation of covalent cross-links, formylation, gamma-carboxylation, glycosylation, glycophosphatidylinositol anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins, arginylation, and ubiquitination.

51. The method according to claim 44, whereby the composition further comprises at least one pharmaceutically acceptable additive, lubricant, diluent, buffer, moistening agent, preservative agent, flavoring, adjuvant, carrier, stabilizer, suspending agent, emulsifying agent, propellant, or other vehicle.

52. A method for preventing cell death in a subject in need thereof comprising, administering to the subject a treatment whereby said treatment induces or suppresses expression of a functional form of ATF6 in an effective amount in the subject.

53. The method according to claim 52, whereby the treatment induces expression a functional form of ATF6.

54. The method according to claim 52, whereby the treatment induces overexpression of a functional form of ATF6.

55. The method according to claim 52, whereby the treatment comprises administering a composition chosen from at least one nucleic acid and at least one protein.

56. The method according to claim 52, whereby the treatment induces expression or overexpression of a functional form of ATF6 by inducing the Unfolded Protein Response.

57. The method according to claim 55, whereby the nucleic acid encodes at least one basal transcription factor that binds to the ER-stress responsive element.

58. The method according to claim 57, whereby the at least one basal transcription factor is chosen from NF-Y, YY1, functional derivatives thereof, and combinations thereof.

59. The method according to claim 55, whereby the nucleic acid comprises at least one transcriptional regulatory sequence.

60. The method according to claim 59, whereby the at least one transcriptional regulatory sequence is chosen from promoters, enhancers, activators, terminators, polyadenylation signals, and combinations thereof.

61. The method according to claim 55, whereby the nucleic acid induces expression of a functional form of ATF6.

62. The method according to claim 61, whereby the nucleic acid sequence that induces expression of a functional form of ATF6 is chosen from Ire1, presenilin 1, preselinin 2, transcription factors of ATF6, site-1 protease, site-2 protease, promoters that replace the original promoter of the gene encoding ATF6 in the cell's genome, enhancers that replace the original promoter of the gene encoding ATF6 in the cell's genome, and combinations thereof.

63. The method according to claim 52, whereby the administering is used as a stand-alone therapy.

64. The method according to claim 55, whereby the nucleic acid comprises a vector chosen from viral vectors, plasmids, and cosmids.

65. The method according to claim 64, whereby the vector is a mammalian vector.

66. The method according to claim 64, whereby the vector directs nucleic acid delivery to the brain.

67. The method according to claim 64, whereby the vector is chosen from neural-specific expression vectors, blood brain barrier transmission-specific vectors, vectors comprising a neural-specific promoter, and combinations thereof.

68. The method according to claim 64, whereby the vector is chosen from retroviruses, adeno-associated viruses, herpes viruses, vaccinia viruses, RNA viruses, herpes simplex virus vectors, adenovirus vectors, adeno-associated virus vectors, lentivirus vectors, vectors comprising a platlet-derived growth factor promoter, vectors comprising a prion promoter, vectors comprising a neuron-specific enolase promoter, vectors comprising promoter/enhancer systems from an immediate early human cytomegalovirus, vectors comprising promoter/enhancer systems from a human neurofilament-light gene, vectors comprising terminators from a cytomegalovirus system, vectors comprising terminators from an SV40 system, and vectors comprising terminators from a bovine growth hormone polyadenylation sequence.

69. The method according to claim 52, whereby the composition further comprises at least one agent that enhances nucleic acid delivery to the brain.

70. The method according to claim 69, whereby the at least one agent that enhances nucleic acid delivery to the brain is mannitol.

71. The method according to claim 52, whereby the composition further comprises at least one pharmaceutically acceptable additive, lubricant, diluent, buffer, moistening agent, preservative agent, flavoring, adjuvant, carrier, stabilizer, suspending agent, emulsifying agent, propellant, or other vehicle.

72. A method for preventing cell death in a cell in need thereof comprising, administering to the subject a treatment whereby said treatment induces or suppresses expression of a functional form of ATF6 in an effective amount in the cell.

73. The method according to claim 72, whereby the treatment induces expression a functional form of ATF6.

74. The method according to claim 72, whereby the treatment induces overexpression of a functional form of ATF6.

75. The method according to claim 72, whereby the treatment comprises administering a composition chosen from at least one nucleic acid and at least one protein.

76. The method according to claim 72, whereby the treatment induces expression or overexpression of a functional form of ATF6 by inducing the Unfolded Protein Response.

77. The method according to claim 75, whereby the nucleic acid encodes at least one basal transcription factor that binds to the ER-stress responsive element.

78. The method according to claim 77, whereby the at least one basal transcription factor is chosen from NF-Y, YY1, functional derivatives thereof, and combinations thereof.

79. The method according to claim 75, whereby the nucleic acid comprises at least one transcriptional regulatory sequence.

80. The method according to claim 79, whereby the at least one transcriptional regulatory sequence is chosen from promoters, enhancers, activators, terminators, polyadenylation signals, and combinations thereof.

81. The method according to claim 75, whereby the nucleic acid comprises a nucleic acid sequence that induces expression of a functional form of ATF6.

82. The method according to claim 81, whereby the nucleic acid sequence that induces expression of a functional form of ATF6 is chosen from Ire1, presenilin 1, preselinin 2, transcription factors of ATF6, site-1 protease, site-2 protease, promoters that replace the original promoter of the gene encoding ATF6 in the cell's genome, enhancers that replace the original promoter of the gene encoding ATF6 in the cell's genome, and combinations thereof.

83. The method according to claim 75, whereby the nucleic acid comprises a vector chosen from viral vectors, plasmids, and cosmids.

84. The method according to claim 83, whereby the vector is a mammalian vector.

85. The method according to claim 83, whereby the vector is chosen from neural-specific expression vectors, blood brain barrier transmission-specific vectors, vectors comprising a neural-specific promoter, and combinations thereof.

86. The method according to claim 83, whereby the vector is chosen from retroviruses, adeno-associated viruses, herpes viruses, vaccinia viruses, RNA viruses, herpes simplex virus vectors, adenovirus vectors, adeno-associated virus vectors, lentivirus vectors, vectors comprising a platlet-derived growth factor promoter, vectors comprising a prion promoter, vectors comprising a neuron-specific enolase promoter, vectors comprising promoter/enhancer systems from an immediate early human cytomegalovirus, vectors comprising promoter/enhancer systems from a human neurofilament-light gene, vectors comprising terminators from a cytomegalovirus system, vectors comprising terminators from an SV40 system, and vectors comprising terminators from a bovine growth hormone polyadenylation sequence.

87. The method according to claim 72, whereby the composition further comprises at least one pharmaceutically acceptable additive, lubricant, diluent, buffer, moistening agent, preservative agent, flavoring, adjuvant, carrier, stabilizer, suspending agent, emulsifying agent, propellant, or other vehicle.

88. A composition comprising at least one viral vector wherein the at least one viral vector comprises nucleic acid encoding a functional form of ATF6.

89. The composition according to claim 88, wherein the at least one functional form of ATF6 is chosen from a full-length form of ATF6, an N-terminal domain form of ATF6, a bZIP-ATF6 fragment, functional derivatives thereof, and combinations thereof.

90. The composition according to claim 89, wherein the full-length form of ATF6 is chosen from human ATF6-α, human ATF6-β, murine ATF6-α, and murine ATF6-β.

91. The composition according to claim 88, wherein the at least one functional form of ATF6 is a full-length form of ATF6 or an N-terminal domain form of ATF6 having at least one modification chosen from deletions, additions, amino acid substitutions, conservative amino acid substitutions, and combinations thereof.

92. The composition according to claim 88, wherein the nucleic acid further comprises a nucleic acid sequence encoding at least one basal transcription factor that binds to the ER-stress responsive element.

93. The composition according to claim 92, wherein the at least one basal transcription factor is chosen from NF-Y, YY1, functional derivatives thereof, and combinations thereof.

94. The composition according to claim 88, wherein the nucleic acid further comprises at least one transcriptional regulatory sequence.

95. The composition according to claim 94, wherein the at least one transcriptional regulatory sequence is chosen from promoters, enhancers, activators, terminators, polyadenylation signals, and combinations thereof.

96. The composition according to claim 88, wherein the nucleic acid further comprises a nucleic acid sequence that induces expression of a functional form of ATF6.

97. The composition according to claim 96, wherein the nucleic acid sequence that induces expression of a functional form of ATF6 is chosen from Ire1, presenilin 1, preselinin 2, transcription factors of ATF6, site-1 protease, site-2 protease, promoters that replace the original promoter of the gene encoding ATF6 in the cell's genome, enhancers that replace the original promoter of the gene encoding ATF6 in the cell's genome, and combinations thereof.

98. The composition according to claim 88, wherein the at least one viral vector is chosen from neural-specific expression vectors, blood brain barrier transmission-specific vectors, vectors comprising a neural-specific promoter, and combinations thereof.

99. The composition according to claim 88, wherein the at least one viral vector is chosen from retroviruses, adeno-associated viruses, herpes viruses, vaccinia viruses, and RNA viruses.

100. The composition according to claim 88, wherein the at least one viral vector directs nucleic acid delivery to the brain.

101. The composition according to claim 88, wherein the at least one viral vector is chosen from neural-specific expression vectors, blood brain barrier transmission-specific vectors, vectors comprising a neural-specific promoter, and combinations thereof.

102. The composition according to claim 88, wherein the at least one viral vector chosen is from retroviruses, adeno-associated viruses, herpes viruses, vaccinia viruses, RNA viruses, herpes simplex virus vectors, adenovirus vectors, adeno-associated virus vectors, lentivirus vectors, vectors comprising a platlet-derived growth factor promoter, vectors comprising a prion promoter, vectors comprising a neuron-specific enolase promoter, vectors comprising promoter/enhancer systems from an immediate early human cytomegalovirus, vectors comprising promoter/enhancer systems from a human neurofilament-light gene, vectors comprising terminators from a cytomegalovirus system, vectors comprising terminators from an SV40 system, and vectors comprising terminators from a bovine growth hormone polyadenylation sequence.

103. The composition according to claim 88, wherein the composition further comprises at least one agent that enhances nucleic acid delivery to the brain.

104. The composition according to claim 103, whereby the at least one agent that enhances nucleic acid delivery to the brain is mannitol.

105. The composition according to claim 88, wherein the composition further comprises at least one pharmaceutically acceptable additive, lubricant, diluent, buffer, moistening agent, preservative agent, flavoring, adjuvant, carrier, stabilizer, suspending agent, emulsifying agent, propellant, or other vehicle.

106. A method for determining whether a substance increases or decreases the activity of at least one functional form of ATF6 in a cell whose production by a cell evokes a responsive change in at least one characteristic, comprising:
providing a first cell line which produces at least one functional form of ATF6;
providing a second cell line which produces the at least one functional form of ATF6 at a lower level than the first cell line, or does not produce the at least one functional form of ATF6 at all;
contacting the substance with the first and second cell lines; and
comparing the at least one characteristic of the first cell lines with the second cell lines.

107. The method according to claim 106, whereby the at least one functional form of ATF6 is chosen from an endogenous form of ATF6, a full-length form of ATF6, an N-terminal domain form of ATF6, a bZIP-ATF6 fragment, functional derivatives thereof, and combinations thereof.

108. The method according to claim 106, whereby the first and second cell lines are neurons.

109. The method according to claim 106, whereby first and second cell lines are nigral neurons.

110. The method according to claim 106, whereby the at least one characteristic is chosen from a characteristic that is observable with the naked eye, a characteristic that is a cultural characteristic of the cell, a characteristic that is a morphological characteristic, and combinations thereof.

111. The method according to claim 106, whereby the at least one characteristic is a measure of an amount of ATF6 mRNA.

112. The method according to claim 106, whereby the at least one characteristic is a measure of an amount of at least one functional form of ATF6.

113. The method according to claim 106, whereby the at least one characteristic is chosen from a rate of cell death, a rate of cell proliferation, and combinations thereof.

114. The method according to claim 106, whereby the at least one characteristic is a response along the UPR signaling pathway.

115. The method according to claim 106, whereby the at least one characteristic is an expression of a gene chosen from Ire1, presenilin 1, presenilin 2, ATF6 regulated genes, and combination thereof.

116. The method according to claim 106, whereby the at least one characteristic is an expression of a UDR-regulated gene.

117. The method according to claim 116, whereby the UDR-regulated gene is chosen from a molecular chaperone BiP gene, a gene involved in protein glycosylation, a gene involved in protein secretion, a gene involved in protein degradation, a gene involved in membrane biosynthesis, and combinations thereof.

118. The method according to claim 106, whereby the at least one characteristic is a cleavage rate of a full length ATF6.

119. The method according to claim 106, whereby the at least one characteristic is a rate of protein folding.

120. The method according to claim 106, whereby the at least one characteristic is a measure of an amount of protein accumulation.

121. The method according to claim 106, whereby the at least one characteristic is chosen from a measure of an amount of protein aggregation, a measure of an amount of protein precipitation, a measure of an amount of protein plaques, and combinations thereof.


SEQUENCE LISTING (11 sequences)

SEQ ID NO: 1 - DNA, 10 bp, human, rat, murine
ggtgacgtgg

SEQ ID NO: 2 - DNA, 10 bp, human, rat, murine
ggtgacgtga

SEQ ID NO: 3 - DNA, 19 bp, human, rat, murine
ccaatcggcggcggccacg

SEQ ID NO: 4 - protein, 670 aa, human
MGEPAGVAGTMESPFSPGLFHRLDEDWDSALFAELGYFTDTDELQLEAANETYENNFDNLDFDLDLLPWESDIWDINNQICTVKDIKAEPQPLSPASSSYSVSSPRSVDSYSSTQHVPEELDLSSSSQMSPLSLYGENSNSLSSPEPLKEDKPVTGSRNKTENGLTPKKKIQVNSKPSIQPKPLLLPAAPKTQTNSSVPAKTIIIQTVPTLMPLAKQQPIISLQPAPTKGQTVLLSQPTVVQLQAPGVLPSAQPVLAVAGGVTQLPNHVVNVVPAPSANSPVNGKLSVTKPVLQSTMRNVGSDIAVLRRQQRMIKNRESACQSRKKKKEYMLGLEARLKAALSENEQLKKENGTLKRQLDEVVSENQRLKVPSPKRRVVCVMIVLAFIILNYGPMSMLEQDSRRMNPSVGPANQRRHLLGFSAKEAQDTSDGIIQKNSYRYDHSVSNDKALMVLTEEPLLYIPPPPCQPLINTTESLRLNHELRGWVHRHEVERTKSRRMTNNQQKTRILQGVVEQGSNSQLMAVQYTETTSSISRNSGSELQVYYASPRSYQDFFEAIRRRGDTFYVVSFRRDHLLLPATTHNKTTRPKMSIVLPAININENVINGQDYEVMMQIDCQVMDTRILHIKSSSVPPYLRDQQRNQTNTFFGSPPAATEATHVVSTIPESLQ

SEQ ID NO: 5 - protein, 703 aa, human
MAELMLLSEIADPTRFFTDNLLSPEDWGLQNSTLYSGLDEVAEEQTQLFRCPEQDVPFDGSSLDVGMDVSPSEPPWELLPIFPDLQVKSEPSSPCSSSSLSSESSRLSTEPSSEALGVGEVLHVKTESLAPPLCLLGDDPTSSFETVQINVIPTSDDSSDVQTKIEPVSPCSSVNSEASLLSADSSSQAFIGEEVLEVKTESLSPSGCLLWDVPAPSLGAVQISMGPSLDGSSGKALPTRKPPLQPKPVVLTTVPMPSRAVPPSTTVLLQSLVQPPPVSPVVLIQGAIRVQPEGPAPSLPRPERKSIVPAPMPGNSCPPEVDAKLLKRQQRMIKNRESACQSRRKKKEYLQGLEARLQAVLADNQQLRRENAALRRRLEALLAENSELKLGSGNRKVVCIMVFLLFIAFNFGPVSISEPPSAPISPRMNKGEPQPRRHLLGFSEQEPVQGVEPLQGSSQGPKEPQPSPTDQPSFSNLTAFPGGAKELLLRDLDQLFLSSDCRHFNRTESLRLADELSGWVQRHQRGRRKIPQRAQERQKSQPRKKSPPVKAVPIQPPGPPERDSVGQLQLYRHPDRSQPAFLDAIDRREDTFYVVSFRRDHLLLPAISHNKTSRPKMSLVMPAMAPNETLSGRGAPGDYEEMMQIECEVMDTRVIHIKTSTVPPSLRKQPSPTPGNATGGPLPVSAASQAHQASHQPLYLNHP

SEQ ID NO: 6 - protein, 678 aa, mouse
LTHPSCEGEVSVSGKPACVAGAMESPFSPVLPHGPDEDWESTLFAELGYFTDTDDVHFDAAHEAYENNFDHLNFDLDLMPWESDLWSPGSHFCSDMKAEPQPLSPASSSCSISSPRSTDSCSSTQHVPEELDLLSSSQSPLSLYGDSCNSPSSVEPLKEEKPVTGPGNKTEHGLTPKKKIQMSSKPSVQPKPLLLPAAPKTQTNASVPAKAIIIQTLPALMPLAKQQSIISIQPAPTKGQTVLLSQPTVVQLQSPAVLSSAQPVLAVTGGAAQLPNHVVNVLPAPVVSSPVNGKLSVTKPVLQSATRSMGSDIAVLRRQQRMIKNRESACQSRKKKKEYMLGLEARLKAALSENEQLKKENGSLKRQLDEVVSENQRLKVPSPKRRAVCVMIVLAFIMLNYGPMSMLEQESRRVKPSVSPANQRRHLLEFSAKEVKDTSDGDNQKDSYSYDHSVSNDKALMVPSEEPLLYMPPPPCQPLINTTESLRLNHELRGWVHRHEVERTKSRRMTNSQQKARILQGALEQGSNSQLMAVQYTETTSISRNSGSELQVYYASPGSYQGFFDAIRRRGDTFYVVSFRRDHLLLPATTHNKTTRPKMSIVLPAININDNVINGQDYEVMMQIDCQVMDTRILHIKSSSVPPYLRDHQRNQTSTFFGSPPTTTETTHVVSTIPESLQ

SEQ ID NO: 7 - protein, 699 aa, murine
MAELMLLSEIADPTRFFTDNLLSPEDWDSTLYSGLDEVAEEQAQLFRCVEQDVPFDSSSLDVGMDVSPPEPPWDPLPIFPDLQVKSEPSSPCSSSSLSSESSHLSTEPPSQVPGVGEVLHVKMESLAPPLCLLGDDPASPFETVQITVGSASDDLSDIQTKLEPASPSSSVHSEASLLSADSPSQPFIGEEVLEVKTESPSPPGCLLWDVPASSLGAVQISMGPSPDSSSGKAPATRKPPLQPKPVVLTTVPVPPRAGPTSAAVLLQPLVQQPAVSPVVLIQGAIRVQPEGPAPAAPRPERKSIVPAPMPGNSCPPEVDAKLLKRQQRMIKNRESACQSRRKKKEYLQGLEARLQAVLADNQQLRRENAALRRRLEALLAENSGLKLGSGNRKVVCIMVFLLFIAFNFGPVSISEPPPAPMSPRMSREEPRPQRHLLGFSEPGPAHGMEPLREAAQSPGEQQPSSAGRPSFRNLTAFPGGAKELLLRDLDQLFLSSDCRHFNRTESLRLADELSGWVQRHQRGRRKIPHRAQERQKSQLRKKSPPVKPVPTQPPGPPERDPVGQLQLYRHPGRSQPEFLDAIDRREDTFYVVSFRRDHLLLPAISHNKTSRPKMSLVMPAMAPNETVSGRGPPGDYEEMMQIECEVMDTRVIHIKTSTVPPSLRKQPSPSPGNTTGGPLPGSAASPAHQASQPLYLNHP

SEQ ID NO: 8 - DNA, 2474 bp, human
aagatattaatcacggagttccagggaaaaggaacttgtgaaatgggggagccggctggggttgccggcaccatggagtcaccttttagcccgggactctttcacaggctggatgaagattgggattctgctctctttgctgaacttggttatttcacagacactgatgagctgcaattggaagcagcaaatgagacgtatgaaaacaattttgataatcttgattttgatttggatttgttaccttgggagtcagacatttgggacatcaacaaccaaatctgtacagttaaagatattaaggcagaaccccagccactttctccagcctcctcaagttattcagtctcatctcctcggtcagtggactcttattcttcaactcagcatgttcctgaggagttggatttgtcttctagttctcagatgtctcccctttccttatatggtgaaaactctaatagtctctcttcaccggagccactgaaggaagataagcctgtcactggttctaggaacaagactgaaaatggactgactccaaagaaaaaaattcaggtgaattcaaaaccttcaattcagcccaagcctttattgcttccagcagcacccaagactcaaacaaactccagtgttccagcaaaaaccatcattattcagacagtaccaacgcttatgccattggcaaagcagcaaccaattatcagtttacaacctgcacccactaaaggccagacggttttgctgtctcagcctactgtggtacaacttcaagcacctggagttctgccctctgctcagccagtccttgctgttgctgggggagtcacacagctccctaatcacgtggtgaatgtggtaccagccccttcagcgaatagcccagtgaatggaaaactttccgtgactaaacctgtcctacaaagtaccatgagaaatgtcggttcagatattgctgtgctaaggagacagcaacgtatgataaaaaatcgagaatccgcttgtcagtctcgcaagaagaagaaagaatatatgctagggttagaggcgagattaaaggctgccctctcagaaaacgagcaactgaagaaagaaaatggaacactgaagcggcagctggatgaagttgtgtcagagaaccagaggcttaaagtccctagtccaaagcgaagagttgtctgtgtgatgatagtattggcatttataatactgaactatggacctatgagcatgttggaacaggattccaggagaatgaaccctagtgtgggacctgcaaatcaaaggaggcaccttctaggattttctgctaaagaggcacaggacacatcagatggtattatccagaaaaacagctacagatatgatcattctgtttcaaatgacaaagccctgatggtgctaactgaagaaccattgctttacattcccccacctccttgtcagcccctaattaatacaacagagtctctcaggttaaatcatgaacttcgaggatgggttcatagacatgaagtagaaaggaccaagtctagaagaatgacaaataatcaacagaaaacccgtattcttcagggtgttgtggaacagggctcaaattctcagctgatggctgttcaatacacagaaaccactagtagtatcagcaggaactcagggagtgagctacaagtgtattatgcttcacccagaagttatcaagacttttttgaagccatccgcagaaggggagacacattttatgttgtgtcatttcgaagggatcacctgctgttaccagctaccacccataacaagaccacaagaccaaaaatgtcaattgtgttaccagcaataaacataaatgagaatgtgatcaatgggcaggactacgaagtgatgatgcagattgactgtcaggtgatggacaccaggatcctccatatcaaaagttcgtcggttcctccttacctccgagatcagcagaggaatcaaaccaacaccttctttggctcccctcccgcagccacagaggcaacccacgttgtcagcaccatccctgagtcattacaatagcacccgcagctatgtggaaaactgagcgtgggacccccagactgaagagcaggtgagcaaaatgctgcttttccttggtggcaggcagagaactgttcgtactagaattcaaggagaaaagaagaagaaataaaagaagctgctccatttttcatcatctacccatctatttggaaagcactggaattcagatgcaagagaacaatgtttcttcagtggcaaatgtagccctgcatcctccagtgttacctggtgtagatttttttttctgtacctttctaaacctctcttccctctgtgatggttttgtgtttaaacagtcatcttcttttaaataatatccacctctcctttttgccatttcacttattgattcataaagtgaattttatttaaagctaaaaaaaaaaaaaaaaaaa

SEQ ID NO: 9 - DNA, 2622 bp, human
aaccgtctcctggttggggggtgggggggaaagatggcggagctgatgctgctcagcgagattgctgacccgacgcgtttcttcaccgacaacctgcttagcccggaggactggggtctgcagaacagcaccttgtattctggcctagatgaagtggccgaggagcagacgcagctcttccgttgcccggagcaggatgtcccgtttgacggcagctccctggacgtggggatggatgtcagcccctctgagcccccatgggaactcctgccgatcttcccagatcttcaggtgaagtctgagccatcttccccctgctcttcctcctccctcagctccgagtcatcgcgtctctccacagagccatccagcgaggctcttggggtaggggaggtgctccatgtgaagacagagtccttggcacccccactgtgtctcctgggagatgacccaacatcctcatttgaaaccgtccagatcaatgttatccccacctctgatgattcctcagatgtccagaccaagatagaacctgtctctccatgttcttccgtcaactctgaggcctccctgctctcagccgactcctccagccaggcttttataggagaggaggtcctggaagtgaagacagagtccctgtccccttcaggatgcctcctgtgggatgtcccagccccctcacttggagctgtccagatcagcatgggcccatcccttgatggctcctcaggcaaagccctgcccacccggaagccgccactgcagcccaaacctgtagtgctaaccactgtcccaatgccatccagagctgtgcctcccagcaccacagtccttctgcagtccctcgtccagccacccccagtgtccccagttgtcctcatccagggtgctattcgagtccagcctgaagggccggctccctctctaccacggcctgagaggaagagcatcgttcccgctcctatgcctggaaactcctgcccgcctgaagtggatgcaaagctgctgaagcggcagcagcgaatgatcaagaaccgggagtcagcctgccagtcccggagaaagaagaaagagtatctgcagggactggaggctcggctgcaagcagtactggctgacaaccagcagctccgccgagagaatgctgccctccggcggcggctggaggccctgctggctgaaaacagcgagctcaagttagggtctggaaacaggaaggtggtctgcatcatggtcttccttctcttcattgccttcaactttggacctgtcagcatcagtgagcctccttcagctcccatctctcctcggatgaacaagggggagcctcaaccccggagacacttgctggggttctcagagcaagagccagttcagggagttgaacctctccaggggtcctcccagggccctaaggagccccagcccagccccacagaccagcccagtttcagcaacctgacagccttccctgggggcgccaaggagctactactaagagacctagaccagctcttcctctcctctgattgccggcacttcaaccgcactgagtccctgaggcttgctgacgagttgagtggctgggtccagcgccaccagagaggccggaggaagatccctcagagggcccaggagagacagaagtctcagccacggaagaagtcacctccagttaaggcagtccccatccaaccccctggacccccagaaagggattctgtgggccagctgcaactatatcgccacccagaccgttcgcagccagcattcttggatgcaattgaccgacgggaagacacattttatgttgtctctttccgaagggaccacctgctgctcccagccatcagccacaacaagacctcccggcccaagatgtccctggtgatgcctgccatggcccccaatgagaccctgtcaggccgtggggccccgggggactatgaggagatgatgcagatcgagtgtgaggtcatggacaccagggtgattcacatcaagacctccacagtgcccccctcgctccgaaaacagccatccccaaccccaggcaatgccacaggtggccccttgccagtctctgcagccagccaggcccaccaggcctcccaccagcccctctacctcaatcatccctgacctctgccattcacactgacttagaacggggggagggggtaccaggtggccaggtgggactgtttcaaatttccctgatccccaggcttggggcaattggtaaaggaaagagcaggtgtgggggttaagcacttatttgaggtgggggtgttcacctctcttctcatcccttttcagaatatagggctcctctcattcctgtgaacccccagtcctggcttctttgtttgaggggattgtgtgaggttcagttgtggggtgggtggtgagctgctgcatattttttattttgtttctctagtgttatggcagtggaggtgggaatttagtccccaggtgggacaagggaagttttttcattttggagctagttactgggagtaagggagggtggggtgggggggagttcaggtttatgtgtgtgcatttcttttttattattattaaataaacaacttggagggagttgaaaaaaaa

SEQ ID NO: 10 - DNA, 4447 bp, murine
ccggagggagaggtgtctgtttcggggaagccggcttgtgttgccggcgccatggagtcgccttttagtccggttcttcctcatggaccagatgaagactgggagtcgacgttgtttgctgaacttggctatttcacagacactgatgatgtgcactttgatgcagcacatgaggcttatgaaaataattttgatcatcttaattttgatttggatttgatgccttgggagtcagacctatggagccccggcagccacttctgctcagacatgaaggcagagccccagcctctttctccggcttcctccagttgctccatctcctctcctcggtccacagactcgtgttcttcaactcagcacgttcctgaggagttggatttgttgtctagttctcagtcccccctttccttatatggcgacagctgtaatagcccctcctctgtagagccactgaaggaagagaagcctgtcactggtcctggaaacaaaacagaacatggactgactccaaagaaaaaaattcagatgagtttaaaaccttcagttcagcccaagcctttattacttccagcagcgcccaagactcaaaccaatgccggtgtcccagcaaaagccatcatcattcagacactaccagcccttatgccactggcaaagcagcagtcgattatcagcatacagcctgcgcccaccaaaggccagactgttttgctctctcagccgactgtggttcaacttcagagccctgcggttctgtcgtctgctcagccggttcttgcagtcactgggggagccgcacagctacctaaccatgtggtgaattgttgctggccagccccctgtggtgagcagcccggtgaatggaaaactttccgtgactaaacctgttctacaaagtgccaccagaagtatgggttcggatatcgctgtgctgaggagacagcagcggatgataaagaaccgagagtctgcttgtcagtcgcgcaagaagaagaaagagtatatgctaggactggaggccaggcctcaaggctgccctctcatagaatgagcagctgtagaaggagaatggctccctgaagcgacagctggacgaggtggtgtcagagaaccagaggctcaaagtcccaagtccaaagcgaagagctgtctgtgtgatgatagtattagcatttataatgctgaactatgggcccatgagcatgctggagcaagaatcccgaagagtgaaacctagtgtgagccctgccaatcagaggaggcatctcttggaattttcagcaaaagaagttaaagacacatcagatggtgacaaccagaaagacagttacagctatgatcactctgtgtccaatgacaaagctttaatggtgctaagtgaagagccattgctttatatgcctccacctccatgtcaacccctgattaacacaacagagtctctcaggttgaaccatgaacttcgaggctgggttcatagacatgaagtggaaaggaccaaatctagaagaatgacaaatagccaacagaaagcccgcattctccagggtgctctggaacagggctctaattctcagctgatggctgtccagtacacagaaaccactagcatcagtaggaattctgggagtgagctgcaagtgtattacgcctcccctggaagttaccaaggcttctttgacgccatccgcaggaggggagatacgttttacgttgtctcatttcgaagggatcatctgctattaccagctaccacccacaacaagaccacaagaccaaaaatgtcaattgtattaccagcaataaacataaatgataatgtgatcaatgggcaggactatgaagtaatgatgcagattgactgtcaggtgatggacaccaggatcctccacatcaaaagctcctcggttcccccttatctccgggatcatcagcggaaccaaaccagcaccttctttggttcccctccaacaaccacagagacgacccatgtggtcagcaccatccctgagtcgttgcagtagtgcccgagctgcgctggacagcagagactgaagagctggtgaagatgctgctctctgcctcttcggcaagcagagacttgccttgtacgcaactccaggggaagaggaagagagaacaggaagtgcgctgcttgtcaccgtccacccagtggggtggaacatgctagcgagcaattctctggtggcagtgcagccctgtgggcagtgtcgcctggtgttggttctgctgtgtcatctttagtgcttttctcaatgtgtgtttggttctcagttatcttccttcaggtcagacccacttcctcttctgtccactgcacttcctggtgcagtaaagagatttgtatttaaagctttagaacacatgctcatgtggtttccaccaattggctttctctctcctttggttcaaatccattctgaatgttatacttgagaaaacacatttcaaaaaaccgagcagccaaaaacatcccacaaagagtcaaaacagtttagagtttgggtaaagggattatctccagttggtaagagtttatttttacttgtgatttgtggttcagccctggacaaataactgttgtgggggtcacagagtgagccacacactggagacaagggaagggaaggccagtggtggaatgtaaggggaagtgactccattttcatatgtatttaaacacagagttcctgtggcctcggtaagctcagagctatagccaccctcagtgttggaactcggctaatcagcagagatcttcaaagatctcagggcacatgcttgcctctcattgtggaccctcagcccagagcatactcctgtgaaaccagactcagcaaagggacttggaggtcactaggcttaagcaagactagagagtttcccttaaggaccaacagtgcacagagcaagcatggcttcccagagaagctgcagcacagtatggtgaagttctcagtttttccagtggaaagatgataaaggaattaagctctctttgttgttgctatggctgtgaacatggctttaatcctagcaccatttggaaggaaaggcaggctttgtttgatatcagcctggcctacatttcaaattccaggacaggacagctaaagctatataaagaacccacctcaaaaaatagatgaatgaataaatgagtaaataaacaaatacaaacaaaaagcaaagttatgttcacatatattttattgtattttgcctgcttccttcaccatagcaagcagccacatttctattgcactgtacattgtacgttacaagttcacagaaatggatgccaggactcatgtcagtcatgtgctgcctcccttcccaggatttcagcaggttctcatagactcttcccagcctggcttgcccattgtcaggtggtcccattccagtaagcacaatggcggctaagtcctcttctctctacaaggagtgacacacagtcaggtcatcttttgcctgtggccccattatgcctggcactgttcaccaacaactgttccctggacagcactgctgccatctaagctaaggtgagatgttttcggggcagggccattcttgctgaattcagtgccgcagtccatcctgattggctctcgggtgattttcagacaagacctgtttgtcccgggggctggtcctctaatgggtgccaaggagaagataccaaatacatggagtacctttaggagtagccatttgtgggggaggttgggctaccctgtggccatgttcttcctgcctgtgaagcagctcaaaacgaggatgtgactgtgggctgtggacagaggcagcacacgcattcctgatgctgatctgctgagacacgaatagaatctgcagtgactccagtgtaccagtgcctcagatcaaagacctcaatagtgtcacgtttgctaaggctgatgcctctcctacaggtaacagtggggatgaccgttggaaggcacagccaaagagcagacagaagttaaggtggccacagcacaggtcagggatccaaggagctggggaggactgctcaaaactagtctggaagcttgccttctctgctcctgctgaccatcaggtcctgtcattaccactctcaggtccgtcttatgagatgaggaatggggccctcctcaggggagagtttcagaaatgagggaaaggcaattatagatagaaagaagtatcctgccatttaaattgctgaaagagctagaatccctgggctcggtagtttgtatcttaatgtttgtgcgctagcacaggcccattggagaggaaaagctgttgtcctgggagcaaagtaagcagccattcaggtctcattttttattttggtatgcttgcccttgggtgtttatagcccggaactgtaggagctatgtatgtacataatatatatattttttaattt

SEQ ID NO: 11 - DNA, 2514 bp, murine
gcggggagccggctcatggtggggggtggggggaagatggcggagctgatgctcctcactgagatcgccgacccgacgcgcttcttcaccgacaacctgctgagtccggaggactgggacagcaccttgtacagtggcctggatgaagtggccgaggagcaggcacagttgttccgttgcgtggagcaggatgtcccgtttgacagcagctctctggatgtggggatggatgtcagcccccctgagcccccttgggaccctctacccatcttcccagatcttcaggtgaagtccgagccatcctctccctgctcgtcctcctccctcagctcagagtcctcacatctttccacagagccccccagccaggtccctggtgtaggcgaggtgctgcatgtgaagatggagtccctggcacccccactctgcctgctgggggatgatccagcatccccctttgaaacggtccagatcactgtgggctctgcctctgatgatctttcagatatccagaccaagctggaacctgcctctccgtcttcttctgtccactctgaggcctccttgctgtcagcagactctcccagtcagccttttataggagaggaggttctggaagtgaagacagagtctccgtcccctccagggtgcctcctgtgggatgtcccagcctcttcgctcggagctgtccagatcagcatgggtccatcccctgatagttcctcagggaaagctccggccactcggaagcctccactgcagcccaagcctgtggtactaaccacagttccggtgccacctagagctgggcctaccagcgctgccgtcctcctgcaacccctggtccagcagcctgcggtgtccccagtggtcctcatccaaggtgctatccgagtccagcctgaagggccagctcccgcagctccccggcctgagaggaagagcattgttccagcccctatgccggggaactcctgcccgcctgaagtggatgcaaagctgttgaagcggcagcagcggatgatcaagaatcgagagtcggcctgccagtcccgccgcaagaagaaagagtacctgccaaggcctggaggccccggctgcaggctgtgctggccgacaaccagcagctgcgcagggagaacgctgccctccggcggcggctggaggccctgctggcagagaacagcgggctcaagctggggtctgggaacaggaaggttgtctgcatcatggtcttccttctcttcattgccttcaacttttggcctgtgagcatcagcgagccgcctccagctcccatgtctcctcggatgagcagggaggaacctcgaccccagaggcacctgctgggcttctcagaaccagggccagctcatggcatggaaccccttcgggaagccgcccagagccccggggagcagcagcccagctctgcaggcaggcccagcttcagaaacctgacggccttccccgggggagccaaggaggctgctgctgagagacctggaccagctcttcctctcctcagactgtcgccatttcaaccgaactgagtctctgaggcttgctgatgagctgagtggctgggtccaacgtcaccagagaggtcgacggaagatacctcacagggcccaggagagacagaagtctcagctacggaagaagtctcctccagtgaaacctgtccccacccaacctccaggaccccctgaaagggaccccgtgggccagctgcagctctaccgccaccccggccgctcgcagccggagtttctagacgcaattgaccggagggaggataccttctatgttgtctccttccgaagggaccacctgctgctcccagccatcagccaccacaagacatccaggcccaagatgtcgctggtgatgccagccatggcccccaatgagaccgtgtcaggccggggccccccaggggactatgaggagatgatgcagatcgagtgtgaggtcatggacaccagggtgattcacatcaagacctctacggtgcccccctcgctccggaagcagccgtccccatccccgggcaataccacaggtggccccttgccaggctccgcagctagtcctgcccatcaggcctcccagcccctttacctcaatcacccctgacatcctcacctcacagtgacttagaaccgggttagggaacctgatcctggggctcgggggcaattgtaaaggaagacggggtgtgggggttaagcacttagtgggactagggtgggtggttcacctctcttctcactctttccagaaatatagggctcctctcattcctgcactcccagtcctctttccccgagggtacctcgtgagggtttcccccatatcctcttcattctctcctttatctgtttgggagtcaaggtgggactaggtcgccaggtgggacaagggatggttgtgggtggcagaagtcagtttatgtgtgtgcgtatcttttttttattattattaaataaacaacgtggaggggtgtaaagg